(19) United States
(12) Patent Application Publication
(10) Pub. No.: US 2025/0237950 A1
(43) Pub. Date: Jul. 24, 2025

(54) RADIATION-SENSITIVE COMPOSITION, METHOD OF FORMING RESIST PATTERN, AND POLYMER

(71) Applicant: JSR CORPORATION, Tokyo (JP)

(72) Inventors: Ken MARUYAMA, Tokyo (JP); Katsuaki NISHIKORI, Tokyo (JP); Kazuya KIRIYAMA, Tokyo (JP)

(73) Assignee: JSR CORPORATION, Tokyo (JP)

(21) Appl. No.: 19/176,934

(22) Filed: Apr. 11, 2025

Related U.S. Application Data
(63) Continuation of application No. PCT/JP2023/036474, filed on Oct. 6, 2023.

(30) Foreign Application Priority Data
Oct. 19, 2022 (JP) 2022-167917

(51) Int. Cl. G03F 7/039; C07C 69/653; C07C 69/78; C07C 309/12; C07C 321/30; C07C 323/09; C08F 212/14; C08F 220/18; C08F 220/22; C08F 220/30

(52) U.S. Cl. CPC G03F 7/0395; C07C 69/653; C07C 69/78; C07C 309/12; C07C 321/30; C07C 323/09; C08F 212/22; C08F 212/24; C08F 220/1807; C08F 220/1812; C08F 220/22; C08F 220/301; C08F 2810/50

(57) ABSTRACT

A radiation-sensitive composition includes a polymer including: an acid-labile side chain including an acid-labile group; and an iodo group-containing side chain including two or more iodo groups and one or more radiation-sensitive onium cation structure(s). A method of forming a resist pattern includes: applying the radiation-sensitive composition directly or indirectly on a substrate to form a resist film; exposing the resist film; and developing the resist film exposed. A polymer includes: an acid-labile side chain including an acid-labile group; and an iodo group-containing side chain including two or more iodo groups and one or more radiation-sensitive onium cation structure(s). A monomer is a vinyl compound including two or more iodo groups and one or more radiation-sensitive onium cation structure(s).

### CROSS-REFERENCE TO RELATED APPLICATIONS

The present application is a continuation application of International Patent Application No. PCT/JP2023/036474 filed Oct. 6, 2023, which claims priority to Japanese Patent Application No. 2022-167917 filed Oct. 19, 2022. The contents of these applications are incorporated herein by reference in their entirety.

### BACKGROUND OF THE DISCLOSURE

### Technical Field

The present disclosure relates to a radiation-sensitive composition, a method of forming a resist pattern, and a polymer.

### Discussion of the Background

A radiation-sensitive composition for use in microfabrication by lithography generates an acid at light-exposed regions upon an irradiation with a radioactive ray, e.g., an electromagnetic wave such as a far ultraviolet ray such as an ArF excimer laser beam (wavelength of 193 nm), a KrF excimer laser beam (wavelength of 248 nm), etc. or an extreme ultraviolet ray (EUV) (wavelength of 13.5 nm), or a charged particle ray such as an electron beam. A chemical reaction in which the acid serves as an origin causes a difference in rates of dissolution in a developer solution of the light-exposed regions and light-unexposed regions, whereby a resist pattern is formed on a substrate.

The radiation-sensitive composition is required not only to have favorable sensitivity to a radioactive ray such as an extreme ultraviolet ray and an electron beam, but also to be superior in CDU (Critical Dimension Uniformity) performance.

Types, molecular structures, and the like of polymers, acid generating agents, and other components which may be used in radiation-sensitive compositions have been investigated to meet these requirements, and combinations thereof have been further investigated in detail (see Japanese Unexamined Patent Application, Publication No. 2010-134279, Japanese Unexamined Patent Application, Publication No. 2014-224984 and Japanese Unexamined Patent Application, Publication No. 2016-047815, and Japanese Unexamined Patent Application, Publication No. 2021-009357).

### SUMMARY

According to an aspect of the present disclosure, a radiation-sensitive composition includes a polymer including: an acid-labile side chain including an acid-labile group; and an iodo group-containing side chain including two or more iodo groups and one or more radiation-sensitive onium cation structure(s).

According to another aspect of the present disclosure, a method of forming a resist pattern includes: applying the radiation-sensitive composition directly or indirectly on a substrate to form a resist film; exposing the resist film; and developing the resist film exposed.

According to a further aspect of the present disclosure, a polymer includes: an acid-labile side chain including an acid-labile group; and an iodo group-containing side chain including two or more iodo groups and one or more radiation-sensitive onium cation structure(s).

According to a further aspect of the present disclosure, a monomer is a vinyl compound including two or more iodo groups and one or more radiation-sensitive onium cation structure(s).

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Along with further microfabrication of resist patterns, required levels for the above-described performance are further elevated, and thus a radiation-sensitive composition that satisfies these requirements are demanded. Particularly, in conventional radiation-sensitive compositions, since an efficiency of acid generation caused by irradiation with a radioactive ray is insufficient, a radiation-sensitive composition having a superior absorption efficiency of a radioactive ray is demanded.

According to one embodiment of the present disclosure, a radiation-sensitive composition contains a polymer (hereinafter, may be also referred to as “(A) polymer” or “polymer (A)”) which includes: a side chain having an acid-labile group; and a side chain having two or more iodo groups and one or more radiation-sensitive onium cation structure(s).

According to another embodiment of the present disclosure, a method of forming a resist pattern includes: applying the above-described radiation-sensitive composition directly or indirectly on a substrate; exposing a resist film formed by the applying; and developing the resist film exposed.

According to still another embodiment of the present disclosure, a polymer includes: a side chain having an acid-labile group; and a side chain having two or more iodo groups and one or more radiation-sensitive onium cation structure(s).

The radiation-sensitive composition of the present disclosure is superior in sensitivity and CDU. The method of forming a resist pattern of the present disclosure enables a resist pattern that is superior in CDU to be formed with favorable sensitivity. The polymer of the present disclosure is suitable as a polymer to be contained in a radiation-sensitive composition. Therefore, these can be suitably used in processing processes of semiconductor devices, and the like, for which microfabrication is expected to progress further hereafter.

The radiation-sensitive composition, the method of forming a resist pattern, and the polymer of the present disclosure are described in detail below.

### Radiation-Sensitive Composition

The radiation-sensitive composition contains the polymer (A). the radiation-sensitive composition typically contains an organic solvent (hereinafter, may be also referred to as “(D) organic solvent” or “organic solvent (D)”). Furthermore, the radiation-sensitive composition preferably contains at least one selected from the group consisting of a radiation-sensitive acid generating agent (hereinafter, may be also referred to as “(B) acid generating agent” or “acid generating agent (B)”) and an acid diffusion control agent (hereinafter, may be also referred to as “(C) acid diffusion control agent” or “acid diffusion control agent (C)”). Moreover, the radiation-sensitive composition may also contain, as a favorable component, a polymer (hereinafter, may be also referred to as “(F) polymer” or “polymer (F)”) having a percentage content of fluorine atoms greater than that of the polymer (A). The radiation-sensitive composition may contain, within a range not leading to impairment of the effects of the present invention, other optional component(s).

Owing to the polymer (A) being contained, the radiation-sensitive composition is superior in sensitivity and CDU. Although not necessarily clarified and without wishing to be bound by any theory, the reason for achieving the aforementioned effects by the radiation-sensitive composition due to involving such a constitution may be presumed, for example, as in the following. It is believed that since an iodo group is highly efficient in absorbing radioactive rays, and the side chain having one or more radiation-sensitive onium cation structure(s) and two or more iodo groups is included, the efficiency of acid generation in light-exposed regions can be improved. It is considered that as a result, the radiation-sensitive composition is superior in sensitivity and CDU.

The radiation-sensitive composition can be prepared by, for example: mixing the polymer (A), and as needed, the radiation-sensitive acid generating agent (B), the acid diffusion control agent (C), the organic solvent (D), the polymer (F), other optional component(s), and the like in a predefined proportion; and preferably filtering a thus obtained mixture through a filter having a pore size of 0.2 m or less.

Each component contained in the radiation-sensitive composition is described below.

### (A) Polymer

The side chain having an acid-labile group included in the polymer (A) is preferably included in a first structural unit (hereinafter, may be also referred to as “structural unit (I)”) which includes a partial structure obtained by substituting a hydrogen atom of a carboxy group or a hydrogen atom of a phenolic hydroxyl group with the acid-labile group. The side chain having two or more iodo groups and one or more radiation-sensitive onium cation structure(s) included in the polymer (A) is preferably included in a second structural unit (hereinafter, may be also referred to as “structural unit (II)”) which includes two or more iodo groups and one or more radiation-sensitive onium cation structure(s). The polymer (A) is a polymer, the solubility of which in a developer solution is capable of being altered by an action of an acid. The polymer (A) may exert the property of alteration of the solubility in a developer solution by the action of an acid, due to having the side chain having an acid-labile group. The radiation-sensitive composition can contain one type, or two or more types of the polymer (A). It is to be noted that as referred to herein, the “structural unit” means one of repeating units obtained by polymerizing a monomer, and is constituted from a portion constituting a part of the main chain, and the side chain. The “main chain” as referred to means the longest atom chain among atom chains that constitute the polymer. The “side chain” as referred to means atom chains other than the main chain, among the atom chains constituting the polymer. In addition, the “partial structure” as referred to herein means a part of a structure included in the side chain or the structural unit.

It is preferred that the polymer (A) further has a side chain having a phenolic hydroxyl group. The side chain having a phenolic hydroxyl group is preferably included in a third structural unit (hereinafter, may be also referred to as “structural unit (III)”) which includes a phenolic hydroxyl group. The polymer (A) may further have other structural unit(s) (hereinafter, may be also referred to as “other structural unit”), aside from the structural units (I) to (III). The polymer (A) can have one type, or two or more types of each structural unit.

The lower limit of a proportion of the polymer (A) contained in the radiation-sensitive composition is, with respect to total components other than the organic solvent (D) contained in the radiation-sensitive composition, preferably 50% by mass, more preferably 70% by mass, and still more preferably 80% by mass. The upper limit of the proportion is preferably 99% by mass, and more preferably 95% by mass.

The lower limit of a polystyrene equivalent weight average molecular weight (Mw) of the polymer (A) as determined by gel permeation chromatography (GPC) is preferably 1,000, more preferably 2,000, and still more preferably 3,000. The upper limit of the Mw is preferably 30,000, more preferably 20,000, and still more preferably 10,000. When the Mw of the polymer (A) falls within the above range, coating characteristics of the radiation-sensitive composition can be improved. The Mw of the polymer (A) can be regulated by adjusting, for example, a type and/or a using amount, etc., of a polymerization initiator to be used in the synthesis.

The upper limit of a ratio (hereinafter, may be also referred to as “Mw/Mn” or “polydispersity”) of Mw to a polystyrene-equivalent number average molecular weight (Mn) of the polymer (A) as determined by GPC is preferably 2.5, more preferably 2.0, and still more preferably 1.7. The lower limit of the ratio is typically 1.0, preferably 1.1, more preferably 1.2, and still more preferably 1.3.

### Methods of Measuring Mw and Mn

As referred to herein, Mw and Mn of the polymer are values measured by using gel permeation chromatography (GPC) under the following conditions.
GPC columns: “G2000 HXL”×2, “G3000 HXL”×1, and “G4000 HXL”×1, available from Tosoh Corporation
column temperature: 40° C.
elution solvent: tetrahydrofuran
flow rate: 1.0 mL/min
sample concentration: 1.0% by mass
amount of injected sample: 100 uL
detector: differential refractometer
standard substance: mono-dispersed polystyrene

The polymer (A) can be synthesized by, for example, polymerizing a monomer that gives each structural unit by a well-known method.

Each structural unit included in the polymer (A) is described below.

### Structural Unit (I)

The side chain having an acid-labile group included in the polymer (A) is preferably included in a structural unit (the first structural unit; hereinafter, may be also referred to as “structural unit (I)”) which includes the partial structure obtained by substituting a hydrogen atom of a carboxy group or a hydrogen atom of a phenolic hydroxyl group with the acid-labile group.

The “acid-labile group” as referred to herein means a group that substitutes for a hydrogen atom in the carboxy group or the phenolic hydroxyl group, and is capable of being dissociated by an action of an acid to give the carboxy group or the phenolic hydroxyl group.

Owing to having the structural unit (I), the polymer (A) enables forming a resist pattern since the acid-labile group is dissociated from the structural unit (I) by an action of the acid generated from the polymer (A) and/or the like upon exposure, whereby a difference is generated in the solubility of the polymer (A) in the developer solution, between light-exposed regions and light-unexposed regions.

The structural unit (I) is not particularly limited as long as it is a structural unit that is dissociated by an action of an acid to give a carboxy group or a phenolic hydroxyl group, and in particular, an acid-labile group (acid-labile group (a-1)) represented by the following formula (1-1), or a structural unit which includes a partial structure substituted with an acid-labile group (acid-labile group (a-2)) represented by the following formula (1-2) is preferred. Hereinafter, the acid-labile group (a-1) and the acid-labile group (a-2) may be collectively referred to as “acid-labile group (a)”. The acid-labile group (a) is a group that substitutes for a hydrogen atom included in the carboxy group or the phenolic hydroxyl group in the structural unit (I). In other words, the acid-labile group (a) is bonded to an etheric oxygen atom of the carbonyloxy group or to an oxygen atom of the phenolic hydroxyl group in the structural unit (I). The “phenolic hydroxy group” as referred to herein means a hydroxy group directly bonding to an aromatic ring in general, without being limited to a hydroxy group directly bonding to a benzene ring.

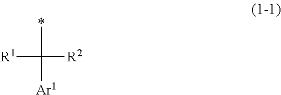

In the above formula (1-1), Ar¹ represents a group obtained by removing one hydrogen atom from a substituted or unsubstituted aromatic ring structure having 5 to 30 ring atoms; R¹ and R² each independently represent a substituted or unsubstituted monovalent aliphatic hydrocarbon group having 1 to 10 carbon atoms, or R¹ and R² taken together represent a saturated alicyclic hydrocarbon ring having 3 to 8 carbon atoms together with the carbon atom to which Ar¹ bonds; and * denotes a binding site to an etheric oxygen atom of a carboxy group or an oxygen atom of a phenolic hydroxyl group.

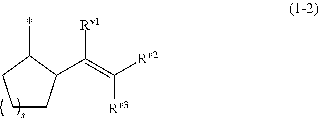

In the above formula (1-2), R^{v1} to R^{v3} each independently represent a hydrogen atom or a substituted or unsubstituted monovalent chain hydrocarbon group having 1 to 10 carbon atoms; s is 1 or 2; and * denotes a binding site to an etheric oxygen atom of a carboxy group or an oxygen atom of a phenolic hydroxyl group.

The number of “ring atoms” as referred to herein means the number of atoms constituting a ring structure, and in the case of a polycyclic ring, the number of “ring atoms” means the number of atoms constituting the polycyclic ring. The “polycyclic ring” may encompass not only a spiro polycyclic ring, in which two rings have one shared atom, and a fused polycyclic ring, in which two rings have two shared atoms, but also a ring-assembled polycyclic ring in which two rings are connected by a single bond without having any shared atom. The “ring structure” may encompass an “alicyclic structure” and an “aromatic ring structure”. The “alicyclic structure” may encompass an “aliphatic hydrocarbon ring structure” and an “aliphatic heterocyclic structure”. Of the alicyclic structures, polycyclic structures such as aliphatic hydrocarbon ring structures and aliphatic heterocyclic structures are defined to fall under the category of the “aliphatic heterocyclic structures”. The “aromatic ring structure” may encompass an “aromatic hydrocarbon ring structure” and an “aromatic heterocyclic structure”. Of the aromatic ring structures, polycyclic structures such as aromatic hydrocarbon ring structures and aromatic heterocyclic structures are defined to fall under the category of the “aromatic heterocyclic structures”. A “group obtained by removing X hydrogen atoms from a ring structure” as referred to means a group obtained by removing X hydrogen atoms bonding to atoms that constitute the ring structure.

The number of “carbon atoms” means the number of carbon atoms constituting a group. The “hydrocarbon group” may encompass an “aliphatic hydrocarbon group” and an “aromatic hydrocarbon group”. The “aliphatic hydrocarbon group” may encompass a “saturated hydrocarbon group” and an “unsaturated hydrocarbon group”. From another viewpoint, the “aliphatic hydrocarbon group” may encompass a “chain hydrocarbon group” and an “alicyclic hydrocarbon group”. The “chain hydrocarbon group” as referred to herein means a hydrocarbon group not having a cyclic structure but being constituted with only a chain structure, and may be exemplified by both a linear hydrocarbon group and a branched hydrocarbon group. The “alicyclic hydrocarbon group” as referred to herein means a hydrocarbon group having, as a cyclic structure, not an aromatic ring structure but an aliphatic ring structure alone, and may be exemplified by both a monocyclic alicyclic hydrocarbon group and a polycyclic alicyclic hydrocarbon group. In this regard, it is not necessary for the alicyclic hydrocarbon group to be constituted with only an aliphatic ring structure, and it may have a chain structure in a part thereof. The “aromatic hydrocarbon group” as referred to herein means a hydrocarbon group which includes an aromatic ring structure as a cyclic structure. In this regard, it is not necessary for the aromatic hydrocarbon group to be constituted with only an aromatic ring structure, and it may have a chain structure or an aliphatic ring structure in a part thereof.

The aromatic ring structure having 5 to 30 ring atoms that gives Ar¹ is exemplified by an aromatic hydrocarbon ring structure having 6 to 30 ring atoms, an aromatic heterocyclic structure having 5 to 30 ring atoms, and the like.

Examples of the aromatic hydrocarbon ring structure having 6 to 30 ring atoms include: a benzene structure; condensed polycyclic aromatic hydrocarbon ring structures such as a naphthalene structure, an anthracene structure, a fluorene structure, a biphenylene structure, a phenanthrene structure, and a pyrene structure; ring-assembled aromatic hydrocarbon ring structures such as a biphenyl structure, a terphenyl structure, a binaphthalene structure, and a phenylnaphthalene structure; and the like.

Examples of the aromatic heterocyclic structure having 5 to 30 ring atoms include: oxygen atom-containing heterocyclic structures such as a furan structure, a pyran structure, a benzofuran structure, and a benzopyran structure; nitrogen atom-containing heterocyclic structures such as a pyrrole structure, a pyridine structure, a pyrimidine structure, an indole structure, and a quinoline structure; sulfur atom-containing heterocyclic structures such as a thiophene structure, and a dibenzothiophene structure; and the like.

The aromatic ring structure having 5 to 30 ring atoms that gives Ar¹ is preferably the aromatic hydrocarbon ring structure having 6 to 30 ring atoms, more preferably a benzene structure or the condensed polycyclic aromatic hydrocarbon ring structure, and still more preferably a benzene structure or a naphthalene structure.

A part or all of hydrogen atom(s) bonding to atom(s) constituting the above-described ring structure may be substituted with a substituent. Examples of the substituent include: halogen atoms such as a fluorine atom and an iodine atom, a hydroxy group, a carboxy group, a cyano group, and a nitro group, as well as alkyl groups described later, fluorinated alkyl groups (groups obtained by substituting a part or all of hydrogen atoms included in an alkyl group, with a fluorine atom), alkoxy groups, alkoxycarbonyl groups, alkoxycarbonyloxy groups, acyl groups, acyloxy groups, an oxo group (═O), and the like. Of these, the halogen atom, the alkyl group, the fluorinated alkyl group or the alkoxy group is preferred, and a fluorine atom, an iodine atom, a methyl group, a trifluoromethyl group, or a methoxy group is more preferred. In the case with a fluorine atom or an iodine atom, sensitivity of the radiation-sensitive composition may be more improved.

Examples of the monovalent aliphatic hydrocarbon group having 1 to 10 carbon atoms that gives each of R¹ and R² include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylprop-1-en-1-yl group; alkynyl groups such as an ethynyl group, a propynyl group, and a butynyl group; and the like.

Examples of the monovalent saturated alicyclic hydrocarbon ring having 3 to 8 carbon atoms which may be represented by R¹ and R² taken together, constituted together with the carbon atom to which Ar¹ bonds include: monocyclic alicyclic saturated hydrocarbon rings such as a cyclopropane ring, a cyclobutane ring, a cyclopentane ring, and a cyclohexane ring; polycyclic alicyclic saturated hydrocarbon rings such as a norbornane ring and an adamantane ring; monocyclic alicyclic unsaturated hydrocarbon rings such as a cyclopentene ring and a cyclohexene ring; polycyclic alicyclic unsaturated hydrocarbon rings such as a norbornene ring; and the like.

The aliphatic hydrocarbon group that gives each of R¹ and R² is preferably a monovalent chain hydrocarbon group having 1 to 10 carbon atoms, or a monovalent alicyclic hydrocarbon group having 3 to 20 carbon atoms, more preferably the alkyl group or a monocyclic alicyclic saturated hydrocarbon group, and still more preferably a methyl group, an ethyl group, an i-propyl group, or a cyclopropyl group.

A part or all of hydrogen atoms in the aliphatic hydrocarbon group may be substituted with a substituent. Examples of the substituent include groups similar to those exemplified as the substituent which may be included in the above-described ring structure that gives Ar¹, and the like. The substituent is preferably a halogen atom or an alkoxy group, and more preferably an iodine atom.

Examples of the alicyclic hydrocarbon ring in the case in which R¹ and R² taken together represent the saturated alicyclic hydrocarbon ring having 3 to 8 carbon atoms together with the carbon atom to which Ar¹ bonds include: monocyclic saturated alicyclic hydrocarbon ring; norbornane rings such as a cyclopropane ring, a cyclobutane ring, a cyclopentane ring, and a cyclohexane ring, polycyclic saturated alicyclic hydrocarbon rings such as an adamantane ring; and the like. Of these, monocyclic saturated alicyclic hydrocarbon rings having 5 or 6 carbon atoms are preferred.

The acid-labile group (a) is preferably a group that substitutes for a hydrogen atom included in the carboxy group, in the structural unit (I), is preferred. In other words, it is preferred that the acid-labile group (a) in the structural unit (I) bonds to an etheric oxygen atom of the carbonyloxy group.

The acid-labile group (a-1) is preferably a group represented by each of the following formulae (a-1-1) to (a-1-24).

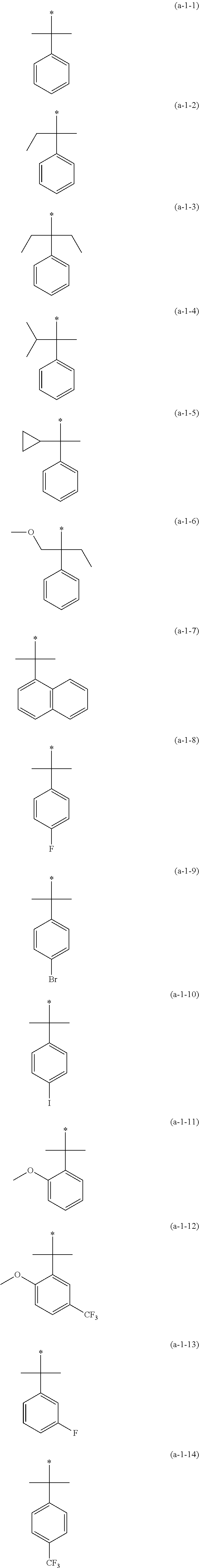

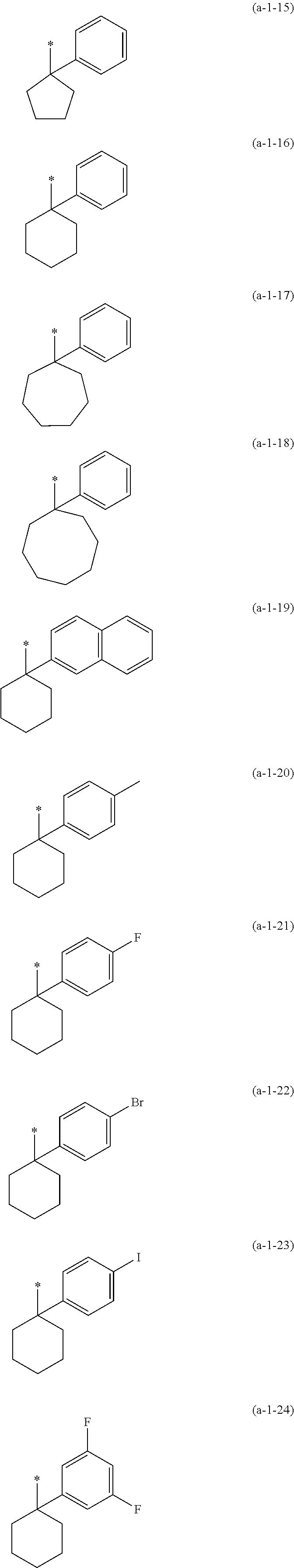

In the above formulae (a-1-1) to (a-1-24), * is as defined in the above formula (1-1).

Examples of the monovalent chain hydrocarbon group having 1 to 10 carbon atoms that gives R^{v1} to R^{v3} include groups similar to those exemplified as the monovalent aliphatic hydrocarbon group having 1 to 10 carbon atoms for R¹ and R².

The acid-labile group (a-2) is preferably a group represented by each of the following formulae (a-2-1) to (a-2-2).

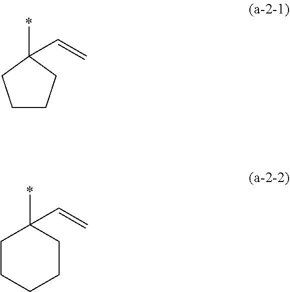

In the above formulae (a-2-1) to (a-2-2), * is as defined in the above formula (1-2).

The structural unit (I) may have an acid-labile group (hereinafter, may be also referred to as “acid-labile group (b)”) other than the acid-labile group (a).

When the polymer (A) has the acid-labile group (b), a balance of sensitivity and CDU can be adjusted.

The acid-labile group (b) is not particularly limited as long as it is a group other than the acid-labile group (a), and examples of the acid-labile group (b) include acid-labile groups (hereinafter, may be also referred to as “acid-labile groups (b-1) to (b-3)”), which are represented by the following formulae (b-1) to (b-3), respectively, and the like.

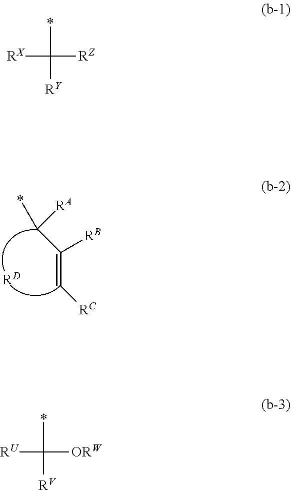

In the above formulae (b-1) to (b-3), * denotes a binding site to an etheric oxygen atom of a carboxy group or an oxygen atom of a phenolic hydroxyl group.

In the above formula (b-1), R^{X} represents a substituted or unsubstituted monovalent saturated aliphatic hydrocarbon group having 1 to 20 carbon atoms, or a substituted or unsubstituted monovalent alicyclic hydrocarbon group having 3 to 20 carbon atoms; and R^{Y} and R^{Z} each independently represent a monovalent aliphatic hydrocarbon group having 1 to 20 carbon atoms, a monovalent alicyclic hydrocarbon group having 3 to 20 carbon atoms, or R^{Y} and R^{Z} taken together represent a saturated alicyclic structure having 3 to 20 ring atoms together with the carbon atom to which R^{Y} and R^{Z} bond.

In the above formula (b-2), R^{A} represents a hydrogen atom; R^{B} and R^{C} each independently represent a hydrogen atom or a monovalent hydrocarbon group having 1 to 20 carbon atoms; and R^{D} represents a divalent hydrocarbon group having 1 to 20 carbon atoms that constitutes an unsaturated alicyclic structure having 4 to 20 ring atoms together with the carbon atom to which R^{A}, R^{B}, and R^{C} each bond.

In the above formula (b-3), R^{U} and R^{V} each independently represent a hydrogen atom or a monovalent hydrocarbon group having 1 to 20 carbon atoms, and R^{W} represents a monovalent hydrocarbon group having 1 to 20 carbon atoms; or R^{U} and R^{V} taken together represent an alicyclic structure having 3 to 20 ring atoms together with the carbon atom to which R^{U} and R^{V} bond, and R^{W} represents a monovalent hydrocarbon group having 1 to 20 carbon atoms; or R^{U} and R^{W} taken together represent an aliphatic heterocyclic structure having 4 to 20 ring atoms together with the carbon atom to which R^{U} bonds and with the oxygen atom to which R^{W} bonds, and R^{V} represents a hydrogen atom or a monovalent hydrocarbon group having 1 to 20 carbon atoms.

Examples of the monovalent aliphatic hydrocarbon group having 1 to 20 carbon atoms which may be represented by R^{Y} or R^{Z} include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylprop-1-en-1-yl group; alkynyl groups such as an ethynyl group, a propynyl group, and a butynyl group.

Examples of a monovalent saturated aliphatic hydrocarbon group having 3 to 20 carbon atoms which may be represented by R^{X} include alkyl groups having 3 to 20 carbon atoms, of the alkyl groups exemplified above.

Examples of the monovalent alicyclic hydrocarbon group having 3 to 20 carbon atoms which may be represented by R^{X}, R^{Y}, or R^{Z} include groups similar to those described for R¹ and R² in connection with the above formula (1-1).

Examples of the saturated alicyclic structure in the case in which R^{Y} and R^{Z} taken together represent a saturated alicyclic structure having 3 to 20 ring atoms together with the carbon atom to which R^{Y} and R^{Z} bond include: monocyclic saturated alicyclic saturated hydrocarbon rings such as a cyclopropane ring, a cyclobutane ring, a cyclopentane ring, and a cyclohexane ring; polycyclic saturated alicyclic saturated hydrocarbon rings such as a norbornane ring and an adamantane ring; and the like.

The monovalent hydrocarbon group having 1 to 20 carbon atoms which may be represented by R^{B}, R^{C}, R^{U}, R^{V}, or R^{W} is exemplified by a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent aliphatic hydrocarbon group having 1 to 20 carbon atoms include groups similar to those described above for R^{X}.

Examples of the monovalent alicyclic hydrocarbon group having 3 to 20 carbon atoms include groups similar to those described for R¹ and R² in connection with the above formula (1-1).

Examples of the monovalent aromatic hydrocarbon group having 6 to 20 carbon atoms include: aryl groups such as a phenyl group, a tolyl group, a xylyl group, a naphthyl group, and an anthryl group; aralkyl groups such as a benzyl group, a phenethyl group, a naphthylmethyl group, and an anthrylmethyl group; and the like.

Examples of the substituent which may be incorporated into the above aliphatic hydrocarbon group represented by R^{X} include groups similar to those exemplified as the substituent which may be included in the above-described ring structure that gives Ar¹ in the formula (1-1) described above, and the like.

Examples of the divalent hydrocarbon group having 1 to 20 carbon atoms represented by R^{D} include groups obtained by removing one hydrogen atom from the groups exemplified as the monovalent hydrocarbon group having 1 to 20 carbon atoms which may be represented by R^{Y}, R^{Z}, R^{B}, R^{C}, R^{U}, R^{V}, or R^{W} described above, and the like.

Examples of the unsaturated alicyclic structure having 4 to 20 ring atoms constituted from R^{D} and the carbon atom to which R^{A}, R^{B}, and R^{C} each bond include: monocyclic unsaturated alicyclic structures such as a cyclobutene structure, a cyclopentene structure, and a cyclohexene structure; polycyclic unsaturated alicyclic structures such as a norbornene structure; and the like.

Examples of the aliphatic heterocyclic structure having 4 to 20 ring atoms represented by R^{U} and R^{W} taken together, constituted together with the carbon atom to which R^{U} bonds and the oxygen atom to which R^{W} bonds include: saturated oxygen-containing heterocyclic structures such as an oxacyclobutane structure, an oxacyclopentane structure, and an oxacyclohexane structure; unsaturated oxygen-containing heterocyclic structures such as an oxacyclobutene structure, an oxacyclopentene structure, and an oxacyclohexene structure; and the like.

In the case in which R^{Y} and R^{Z} each represent the monovalent hydrocarbon group having 1 to 20 carbon atoms, R^{Y} and R^{Z} are each preferably a chain hydrocarbon group, more preferably the alkyl group, and still more preferably a methyl group. R^{X} in this case represents preferably the chain hydrocarbon group, more preferably the alkyl group, and still more preferably a methyl group.

In the case in which R^{Y} and R^{Z} taken together represent the saturated alicyclic structure having 3 to 20 ring atoms together with the carbon atom to which R^{Y} and R^{Z} bond, the saturated alicyclic structure is preferably a monocyclic saturated alicyclic structure, and more preferably a cyclopentane structure or a cyclohexane structure. R^{X} in this case in preferably a chain hydrocarbon group, more preferably the alkyl group, and still more preferably a methyl group, an ethyl group, an i-propyl group, or a tert-butyl group.

The case in which R^{Y} and R^{Z} taken together represent the saturated alicyclic structure having 3 to 20 ring atoms together with the carbon atom to which R^{Y} and R^{Z} bond is preferred. In this case, CDU of the radiation-sensitive composition can be more improved.

R^{B} represents preferably a hydrogen atom.

R^{C} represents preferably a hydrogen atom or the chain hydrocarbon group, more preferably a hydrogen atom or the alkyl group, and still more preferably a methyl group.

The unsaturated alicyclic structure having 4 to 20 ring atoms constituted from R^{D} together with the carbon atom to which R^{A}, R^{B}, and R^{C} each bond is preferably the monocyclic unsaturated alicyclic structure, and more preferably a cyclopentane structure or a cyclohexene structure.

The acid-labile group (b) is preferably an acid-labile group (b-1) or (b-2).

Examples of the acid-labile group (b-1) include groups represented by the following formulae (b-1-1) to (b-1-13), and the like. Examples of the acid-labile group (b-2) include groups represented by the following formulae (b-2-1) to (b-2-2), and the like.

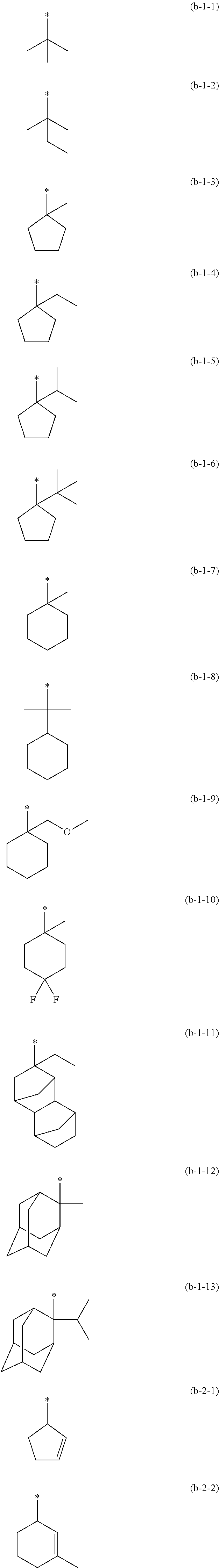

In the above formulae (b-1-1) to (b-1-13) and (b-2-1) to (b-2-2), * is as defined in the above formulae (b-1) and (b-2).

Examples of the structural unit (I) include a structural unit (hereinafter, may be also referred to as “structural unit (I-1) or (I-2)”) represented by the following formula (3-1) or (3-2), and the like.

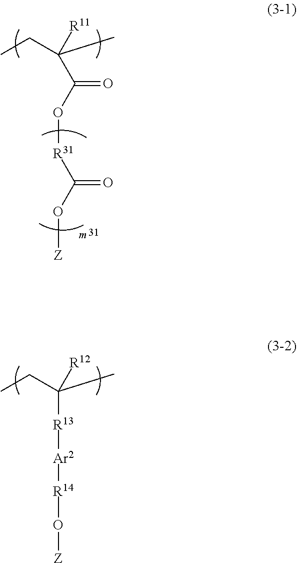

In the above formulae (3-1) and (3-2), Z represents the acid-labile group. Z is preferably an acid-labile group (acid-labile group (a-1) or acid-labile group (a-2)) represented by the above formula (1-1) or (1-2), or the acid-labile group (acid-labile group (b-1) to (b-2)) represented by the above formulae (b-1) to (b-2).

In the above formula (3-1), R¹¹ represents a hydrogen atom, a fluorine atom, a methyl group, or a trifluoromethyl group; R³¹ represents a divalent linking group; and m³¹ is 0 or 1.

The divalent linking group in R³¹ is exemplified by groups similar to those exemplified as the divalent linking group represented by L^{s} and Q^{s} described later. Of those, a divalent hydrocarbon group having 1 to 10 carbon atoms is preferred, and an alkylene group is more preferred.

In the above formula (3-2), R¹² represents a hydrogen atom or a methyl group; R¹³ represents a single bond, an oxygen atom, —COO—, or —CONH—; Ar² represents a group obtained by removing two hydrogen atoms from a substituted or unsubstituted aromatic hydrocarbon ring structure having 6 to 30 ring atoms; and R¹⁴ represents a single bond or —CO—.

R¹¹ represents, in light of a degree of copolymerization of a monomer that gives the structural unit (I), preferably a hydrogen atom or a methyl group, and more preferably a methyl group.

R¹³ represents preferably a single bond.

The aromatic hydrocarbon ring structure having 6 to 30 ring atoms that gives Ar² is exemplified by, among the aromatic ring structures having 5 to 30 ring atoms that can give Ar¹ in the above formula (1-1), ring structures similar to those exemplified as the aromatic hydrocarbon ring structure having 6 to 30 ring atoms; and the like. Of these, a benzene structure or a naphthalene structure is preferred.

R¹⁴ represents preferably a single bond.

The structural unit (I) is preferably the structural unit (I-1).

The lower limit of a proportion of the structural unit (I) contained in the polymer (A) with respect to the total structural units constituting the polymer (A) is preferably 5 mol %, more preferably 15 mol %, still more preferably 20 mol %, and particularly preferably 25 mol %. The upper limit of the proportion is preferably 70 mol %, more preferably 60 mol %, still more preferably 50 mol %, and particularly preferably 40 mol %. When the proportion of structural unit (I) falls within the above range, the sensitivity and CDU of the radiation-sensitive composition can be more improved. With respect to descriptions of the upper limit and the lower limit of numerical ranges as referred to herein, unless otherwise specified particularly, the upper limit may have the meaning of either “no greater than” or “less than”, and the lower limit may have the meaning of either “no less than” or “greater than”. Further, the upper limit value and the lower limit value may be combined ad libitum.

The lower limit of a proportion of the structural unit having the acid-labile group (a), of the structural units (I) in the polymer (A), is 0 mol %, preferably 15 mol %, more preferably 30 mol %, still more preferably 45 mol %, even more preferably 60 mol %, and particularly preferably 75 mol %, with respect to the content of the structural unit (I). The upper limit of the proportion is, with respect to the content of the structural unit (I), 100 mol %, preferably 85 mol %, more preferably 70 mol %, still more preferably 55 mol %, even more preferably 40 mol %, and particularly preferably 25 mol %.

The lower limit of a proportion of the structural unit having the acid-labile group which includes iodo group(s), of the structural units (I) in the polymer (A), is 0 mol %, preferably 15 mol %, more preferably 30 mol %, still more preferably 45 mol %, even more preferably 60 mol %, and particularly preferably 75 mol %, with respect to the content of the structural unit (I). The upper limit of the proportion is, with respect to the content of the structural unit (I), 100 mol %, preferably 85 mol %, more preferably 70 mol %, still more preferably 55 mol %, even more preferably 40 mol %, and particularly preferably 25 mol %.

The polymer (A) having the structural unit (I) can be synthesized by polymerize a monomer that gives a structural unit (I) by a well-known method.

### Structural Unit (II)

The side chain having two or more iodo groups and one or more radiation-sensitive onium cation structure(s) included in the polymer (A) is preferably included in a structural unit (the second structural unit, may be also referred to as “structural unit (II)”) which includes two or more iodo groups and one or more radiation-sensitive onium cation structure(s). Also, the structural unit (II) may be mentioned as a structural unit which includes a partial structure that generates an acid upon irradiation with a radioactive ray (hereinafter, may be also referred to as “exposure”).

The number of the iodo groups in the structural unit (II) may be two or more, preferably two to sic, more preferably two to four, and still more preferably two or three.

At least one of the iodo groups in the structural unit (II) is preferably bonded to an aromatic ring structure. The aromatic ring structure is exemplified by ring structures similar to those exemplified as the aromatic ring structure having 5 to 30 ring atoms that gives Ar¹ in the above formula (1-1). Of these, the aromatic hydrocarbon ring structure having 6 to 30 ring atoms is preferred, the aromatic hydrocarbon ring structure having 6 to 10 ring atoms is more preferred, and a benzene ring is still more preferred. Note that it is not necessary for two or more iodo groups to be bonded to an identical aromatic ring structure, and two or more aromatic ring structures to which one iodo group each bonds may be included.

The structural unit (II) is exemplified by: a structure (hereinafter, may be also referred to as “structure 1”) which includes a sulfonate anion and a radiation-sensitive onium cation, wherein the sulfonate anion is bonded to the side chain of the polymer; and a structure (hereinafter, may be also referred to as “structure 2”) which includes a sulfonate anion and a radiation-sensitive onium cation, wherein the radiation-sensitive onium cation is bonded to the side chain of the polymer. Of these, the structure 1 is preferred.

The radiation-sensitive onium cation is exemplified by onium cations similar to those exemplified as the radiation-sensitive onium cation in a photodegradable base to be served as the acid generating agent (B) and/or the acid diffusion control agent (C), as described later. Of these, a sulfonium cation is preferred, and a monovalent radiation-sensitive sulfonium cation which includes an aromatic ring structure having at least one selected from the group consisting of a fluorine atom, a fluorine atom-containing group, and an iodine atom is preferred. Specific modes and preferred modes are incorporated here by reference of a description regarding the radiation-sensitive onium cation referred to in the description of (B) Acid Generating Agent as described later.

The structural unit (II) is preferably the structure 1, and examples thereof include structural units which each include a partial structure represented by the following formula (II-0), and the like.

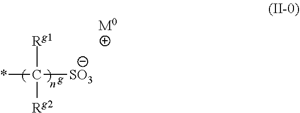

In the above formula (II-0), R^{g1} and R^{g2} each independently represent a fluorine atom or a monovalent fluorinated hydrocarbon group having 1 to 20 carbon atoms; n^{g} is an integer of 1 to 10; M⁰⁺ represents a monovalent radiation-sensitive onium cation; and * denotes an atomic bonding with an other partial structure, in the structural unit (II).

The monovalent fluorinated hydrocarbon group having 1 to 20 carbon atoms which may be represented by each of R^{g1} and R^{g2} is exemplified by a fluorinated alkyl group having 1 to 20 carbon atoms; and the like. R^{g1} and R^{g2} each represent preferably a fluorine atom or a fluorinated alkyl group having one to six carbon atoms, more preferably a fluorine atom or a perfluoroalkyl group having one to six carbon atoms, still more preferably a fluorine atom or a trifluoromethyl group, and particularly preferably a fluorine atom.
n^{g} is preferably 1 to 4, and more preferably 1 or 2.

The structural unit (II) can be obtained by polymerizing: a (meth)acrylic acid ester compound (hereinafter, may be also referred to as “compound (II-1)”) which includes two or more iodo groups and one or more radiation-sensitive onium cation structure(s); or a vinyl compound (hereinafter, may be also referred to as “compound (II-2)”) which includes two or more iodo groups and one or more radiation-sensitive onium cation structure(s).

The compound (II-1) is exemplified by
a monomer that is a salt (hereinafter, may be also referred to as “monomer (II-1-1)”), which includes: a sulfonate anion having a (meth)acryloyloxy group and two or more iodo groups; and a radiation-sensitive onium cation,
a monomer that is a salt (hereinafter, may be also referred to as “monomer (II-1-2)”), which includes: a sulfonate anion having a (meth)acryloyloxy group and one iodo group; and a radiation-sensitive onium cation having one iodo group; and
a monomer that is a salt (hereinafter, may be also referred to as “monomer (II-1-3)”), which includes: a sulfonate anion having a (meth)acryloyloxy group; and a radiation-sensitive onium cation having two or more iodo groups. Of these, the monomer (II-1-1) is preferred.

The sulfonate anion in the monomer (II-1-1) may be exemplified by: a sulfonate anion having an aromatic ring to which two or more iodo groups bond, and one (meth)acryloyloxy group; and a sulfonate anion having two or more aromatic rings to which one iodo group bonds, and one (meth)acryloyloxy group. The upper limit of a proportion of the iodine atom contained in these sulfonate anions is, based on the molecular weight of the sulfonic acid in which a proton bonds to the sulfonate anion, preferably no greater than 50%, more preferably no greater than 45%, still more preferably no greater than 40%, and particularly preferably no greater than 35%. Further, the lower limit of the proportion is preferably no less than 10%, more preferably no less than 20%, and still more preferably no less than 25%.

The compound (II-2) is exemplified by
a monomer that is a salt (hereinafter, may be also referred to as “monomer (II-2-1)”), which includes: a sulfonate anion having a vinyl group and two or more iodo groups; and a radiation-sensitive onium cation,
a monomer that is a salt (hereinafter, may be also referred to as “monomer (II-2-2)”), which includes: a sulfonate anion having a vinyl group and one iodo group; and a radiation-sensitive onium cation having one iodo group; and
a monomer that is a salt (hereinafter, may be also referred to as “monomer (II-2-3)”), which includes: a sulfonate anion having a vinyl group; and a radiation-sensitive onium cation having two or more iodo groups. Of these, the monomer (II-2-1) is preferred.

The sulfonate anion in the monomer (II-2-1) may be exemplified by: a sulfonate anion having an aromatic ring to which two or more iodo groups bond, and one vinyl group; and a sulfonate anion having two or more aromatic rings to which one iodo group bonds, and one vinyl group. The upper limit of a proportion of the iodine atom contained in these sulfonate anions is, based on the molecular weight of the sulfonic acid in which a proton bonds to the sulfonate anion, preferably no greater than 50%, more preferably no greater than 45%, still more preferably no greater than 40%, and particularly preferably no greater than 35%. Further, the lower limit of the proportion is preferably no less than 10%, more preferably no less than 20%, still more preferably no less than 25%, and particularly preferably no less than 30%.

Examples of the compound (II-1) include a compound represented by the following formula (II-1s), and the like.

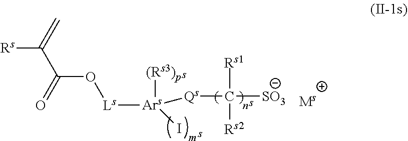

In the above formula (II-1s), R^{s} represents a hydrogen atom or a methyl group; L^{s} and Q^{s} each represent a single bond or a divalent linking group; Ar^{s} represents an aromatic hydrocarbon group having 6 to 20 carbon atoms and having a valency of (m^{s}+p^{s}+2); R^{s1} and R^{s2} each independently represent a fluorine atom or a monovalent fluorinated hydrocarbon group having 1 to 20 carbon atoms; R^{s1} represents a substituent other than an iodo group; m^{s} is an integer of 0 to 4; n^{s} is an integer of 1 to 10; p^{s} is an integer of 0 or more; and M^{s+} represents a monovalent radiation-sensitive onium cation, wherein in the case in which m^{s} is 0, L^{s} includes an aromatic ring having two or more iodo groups, or includes two or more aromatic rings having one iodo group, or wherein in the case in which m^{s} is 1, L^{s} includes an aromatic ring having one or more iodo group(s).

Examples of the divalent linking group which may be represented by each of L^{s} and Q^{s} include a carbonyl group, an ether group, a carbonyloxy group, a sulfide group, a thiocarbonyl group, a sulfonyl group, a divalent hydrocarbon group or a group obtained by combining the same; and the like. The carbon atom constituting the divalent hydrocarbon group may be replaced with a carbonyl group or an ether group. L^{s} represents preferably a single bond or a divalent hydrocarbon group having 1 to 10 carbon atoms. Q^{s} represents preferably a group obtained by combining one or more type(s) selected from the group consisting of a carbonyl group, an ether group, a carbonyloxy group, and a divalent hydrocarbon group having 1 to 20 carbon atoms, wherein the carbon atom constituting the divalent hydrocarbon group having 1 to 20 carbon atoms may be replaced with a carbonyl group or an ether group.

It is to be noted that in the case in which m^{s} is 0, L^{s} includes an aromatic ring having two or more iodo groups, or includes two or more aromatic rings having one iodo group. Alternatively, in the case in which m^{s} is 1, L^{s} includes an aromatic ring having one or more iodo group(s). Examples of such an aromatic ring having iodo group(s) include an iodophenylene group, an iodotolylene group, an iodonaphthylene group, a diiodophenylene group, a diiodonaphthylene group, and the like. The aromatic ring may further have a substituent, and examples of such a substituent include a fluoro group, a chloro group, a bromo group, an alkoxy group, a hydroxy group, a carboxy group, a nitro group, and the like.

Examples of the aromatic hydrocarbon ring having 6 to 20 carbon atoms that gives the aromatic hydrocarbon group having 6 to 20 carbon atoms and having a valency of (m^{s}+p^{s}+2), represented by Ar^{s} include: a benzene ring; condensed polycyclic aromatic hydrocarbon rings such as a naphthalene ring, an anthracene ring, a fluorene ring, a biphenylene ring, a phenanthrene ring, and a pyrene ring; ring-assembled aromatic hydrocarbon rings such as a biphenyl ring, a terphenyl ring, a binaphthalene ring, and a phenylnaphthalene ring; a 9,10-ethanoanthracene ring; a triptycene ring; and the like. Of these, a benzene ring, and a naphthalene ring are preferred. Ar^{s} may have a substituent, and examples of such a substituent include a halogen atom, an alkoxy group, a hydroxy group, a carboxy group, a nitro group, and the like.

The monovalent fluorinated hydrocarbon group having 1 to 20 carbon atoms which may be represented by each of R^{s1} and R^{s2} is exemplified by a fluorinated alkyl group having 1 to 20 carbon atoms, and the like. R^{s1} and R^{s2} each represent preferably a fluorine atom or a fluorinated alkyl group, more preferably a fluorine atom or a perfluoroalkyl group, still more preferably a fluorine atom or a trifluoromethyl group, and particularly preferably a fluorine atom.

Examples of the substituent, other than an iodo group, represented by R^{S3} include a fluoro group, a chloro group, a bromo group, an alkoxy group, a hydroxy group, a carboxy group, a nitro group, and the like.
m^{s} is preferably 2 or 3.
n^{s} is preferably 1 to 4, and more preferably 1 or 2.
M^{s+} is exemplified by those similar to radiation-sensitive onium cations exemplified later.

The compound (II-1) is preferably a compound represented by each of the following formulae (II-1-1) to (II-1-10).

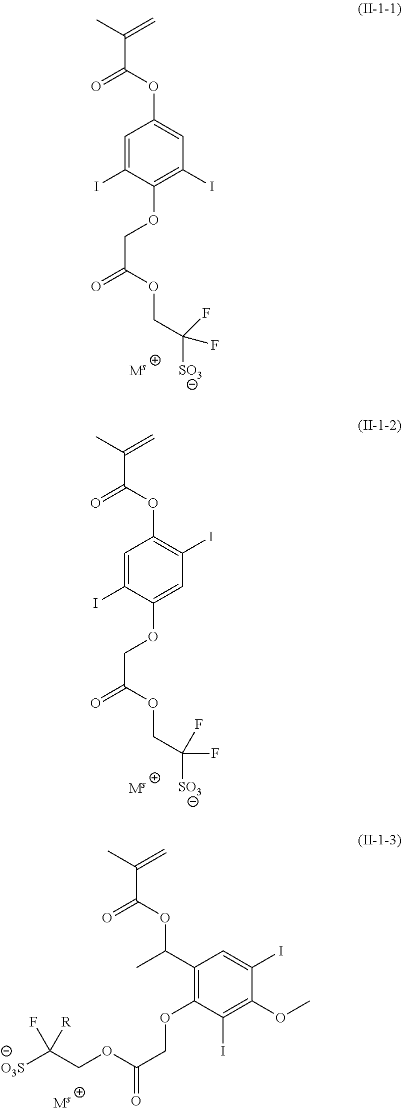

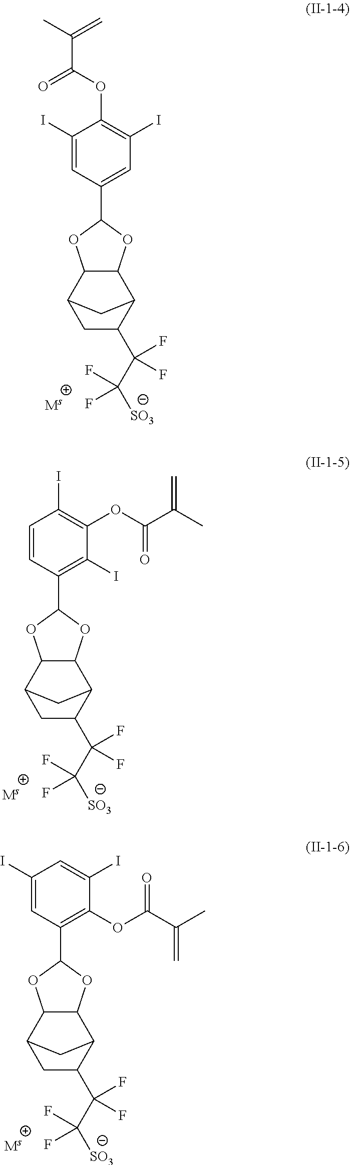

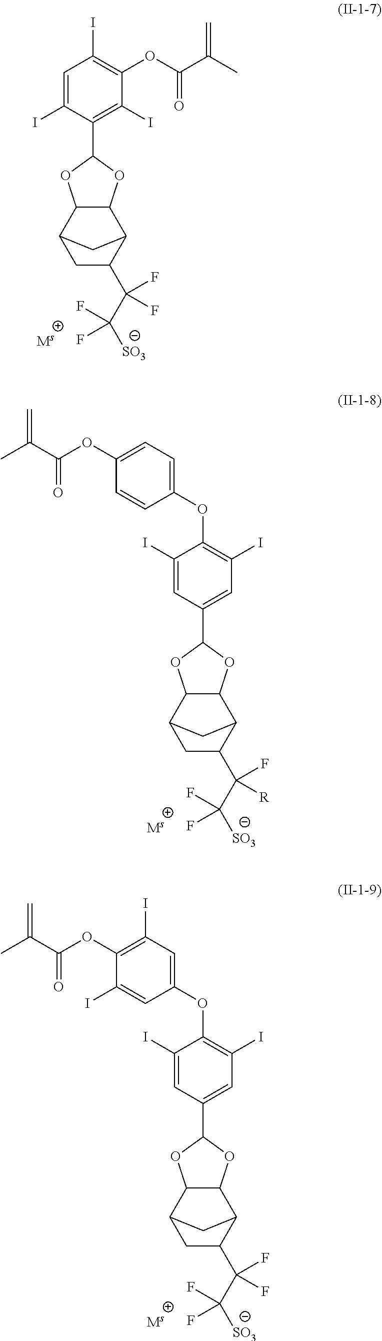

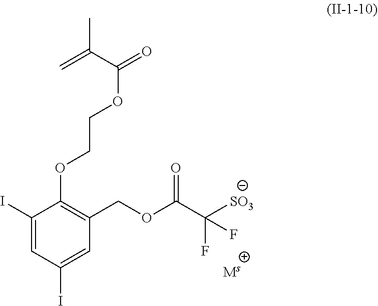

In the above formulae (II-1-1) to (II-1-10), M^{s+} is as defined in the above formula (II-1s).

Examples of the compound (II-2) include a compound represented by the following formula (II-2t) or formula (II-2u), and the like.

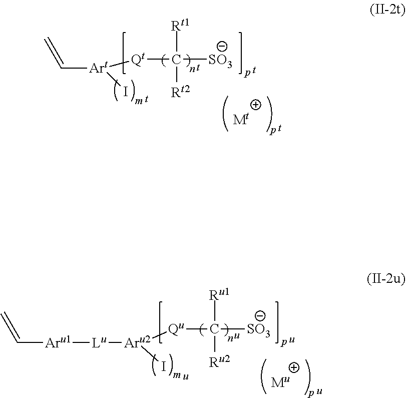

In the above formula (II-2t), Q^{t} represents a single bond or a divalent linking group; Ar^{t} represents an aromatic hydrocarbon group having 6 to 20 carbon atoms and having a valency of (m^{t}+pt+1); R^{t1} and R^{t2} each independently represent a fluorine atom or a monovalent fluorinated hydrocarbon group having 1 to 20 carbon atoms; m^{t} is an integer of 2 to 4; n^{t} is an integer of 1 to 10; p^{t} is 1 or 2; and M^{t+} represents a monovalent radiation-sensitive onium cation, wherein: in the case in which p^{t} is 2, two Q^{t}s are identical or different, and two M^{t+}s are identical or different; in the case in which n^{t} is no less than 2 or p^{t} is 2, a plurality of R^{t1}s and R^{t2}s being present are each independently identical or different.

In the above formula (II-2u), L^{u} represents a divalent linking group; Q^{u} represents a single bond or a divalent linking group; Ar^{u1} represents a divalent aromatic hydrocarbon group having 6 to 20 carbon atoms; Ar^{u2} represents an aromatic hydrocarbon group having 6 to 20 carbon atoms and having a valency of (m^{u}+p^{u}+1); R^{u1} and R^{u2} each independently represent a fluorine atom or a monovalent fluorinated hydrocarbon group having 1 to 20 carbon atoms; m^{u} is an integer of 0 to 4; n^{u} is an integer of 1 to 10; p^{u} is 1 or 2; and M^{u+} represents a monovalent radiation-sensitive onium cation, wherein: in the case in which p^{u} is 2, two Q^{u}s are identical or different, two M^{u+}s are identical or different; and in the case in which n^{u} is no less than 2 or p^{u} is 2, a plurality of R^{u1}s and R^{u2}s being present are each independently identical or different; however, in the case in which m^{u} is 0, Ar^{u1} includes an aromatic ring having two or more iodo groups, or includes two or more aromatic rings having one iodo group; and in the case in which m^{u} is 1, Ar^{u1} includes an aromatic ring having one or more iodo group(s).

Examples of the divalent linking group which may be represented by each of Q^{t}, L^{u} and Q^{u} include a carbonyl group, an ether group, a carbonyloxy group, a sulfide group, a thiocarbonyl group, a sulfonyl group, a divalent hydrocarbon group, and the like. The carbon atom constituting divalent hydrocarbon group may be replaced with a carbonyl group or an ether group. L^{u} represents preferably a carbonyloxy group. Q^{t} and Q^{u} represent preferably a group obtained by combining one or more type(s) selected from the group consisting of a carbonyl group, an ether group, a carbonyloxy group, and a divalent hydrocarbon group having 1 to 20 carbon atoms, wherein the carbon atom constituting the divalent hydrocarbon group having 1 to 20 carbon atoms may be replaced with a carbonyl group or an ether group.

It is to be noted that in the case in which m^{u} is 0, Ar^{u1} includes an aromatic ring having two or more iodo groups, or includes two or more aromatic rings having one iodo group. Alternatively, in the case in which m^{u} is 1, Ar^{u1} includes an aromatic ring having one or more iodo group(s). Examples of such an aromatic ring having iodo group(s) include an iodophenylene group, an iodotolylene group, an iodonaphthylene group, a diiodophenylene group, a diiodonaphthylene group, and the like.

The aromatic hydrocarbon group having 6 to 20 carbon atoms and having a valency of (m^{t}+p^{t}+1), represented by Ar^{t}, the divalent aromatic hydrocarbon group having 6 to 20 carbon atoms represented by Ar^{u1}, and the aromatic hydrocarbon ring having 6 to 20 carbon atoms that gives the aromatic hydrocarbon group having 6 to 20 carbon atoms and having a valency of (m^{u}+p^{u}+1), represented by Ar^{u2} are exemplified similarly to those exemplified as the above-described aromatic hydrocarbon ring having 6 to 20 carbon atoms that gives the aromatic hydrocarbon group having 6 to 20 carbon atoms and having a valency of (m^{s}+p^{s}+2), represented by Ar⁶. Of these, a benzene ring and a naphthalene ring are preferred. Ar^{t}, Ar^{u1} and Ar^{u2} may have a substituent, and examples of such a substituent include a halogen atom, an alkoxy group, a hydroxy group, a carboxy group, a nitro group, and the like.

The monovalent fluorinated hydrocarbon group having 1 to 20 carbon atoms which may be represented by each of R^{t1}, R^{t2}, R^{u1}, and R^{u2} is exemplified by a fluorinated alkyl group having 1 to 20 carbon atoms, and the like. R^{s1} and R^{s2} each represent preferably a fluorine atom or a fluorinated alkyl group, more preferably a fluorine atom or a perfluoroalkyl group, still more preferably a fluorine atom or a trifluoromethyl group, and particularly preferably a fluorine atom.
m^{t} is preferably 2 or 3. m^{u} is preferably 1 to 3, and more preferably 2 or 3.
n^{t} and n^{u} are each preferably 1 to 4, and more preferably 1 or 2.
M^{t+} and M^{u+} are exemplified by those similar to radiation-sensitive onium cations exemplified later.

The compound (II-2) is preferably any one of compounds represented by the following formulae (II-2-1) to (II-2-17). It is to be noted that the compounds represented by the following formula (II-2-9) and formula (II-2-17) do not fall under both the compounds represented by the above formula (II-2t) and formula (II-2u).

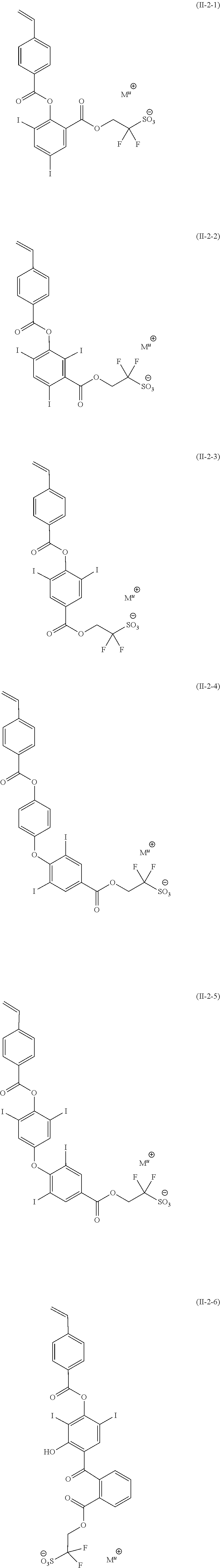

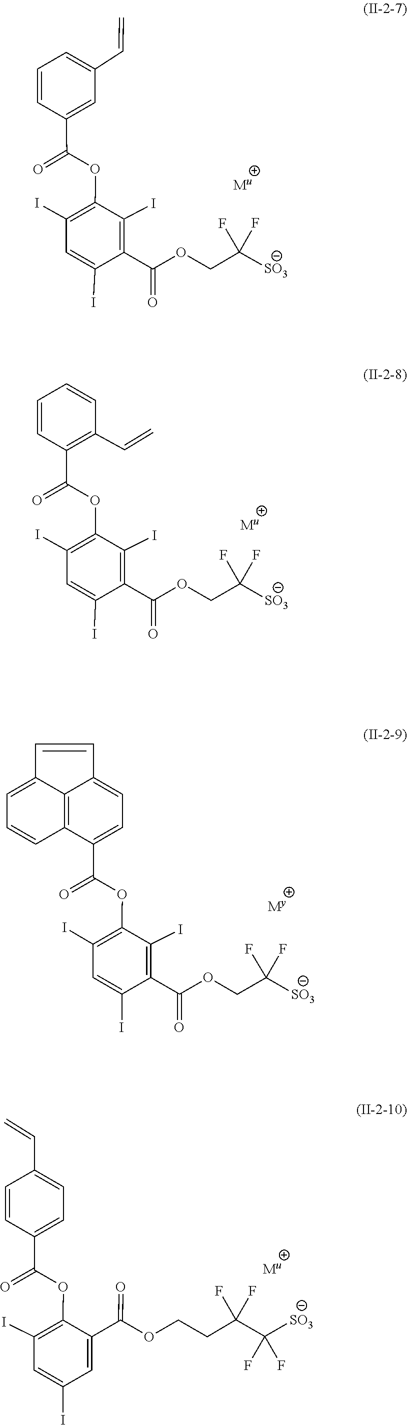

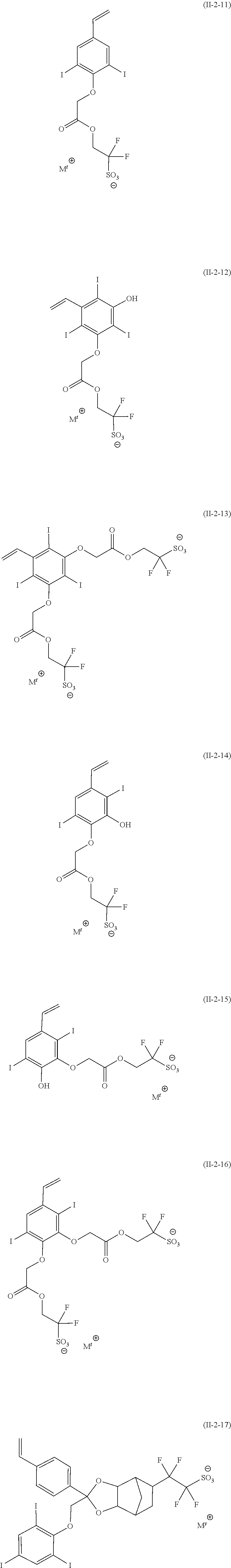

In the above formulae (II-2-1) to (II-2-8) and (II-2-10), M^{u+} is as defined in the above formula (II-2u). In the above formulae (II-2-11) to (II-2-16), M^{t+} is as defined in the above formula (II-2t). In the above formulae (II-2-9) and (II-2-17), M^{y+} represents a monovalent radiation-sensitive onium cation. It is to be noted that two M^{t+} in the above formulae (II-2-13) and (II-2-16) are each independent.

The case in which the compound (II-2) is used as the structural unit (II) is preferred since CDU may be favorable. Although the reason for this feature is not certain, the inventor considers that it may be related to a glass transition temperature of the polymer (A).

The lower limit of a proportion of the structural unit (II) contained in the polymer (A) is, with respect to the total structural units constituting the polymer (A), preferably 1 mol %, more preferably 3 mol %, still more preferably 5 mol %, and particularly preferably 7 mol %. The upper limit of the proportion is preferably 40 mol %, more preferably 30 mol %, and still more preferably 20 mol %. When the proportion of the structural unit (II) falls within the above range, the sensitivity and CDU of the radiation-sensitive composition can be more improved.

### Structural Unit (III)

It is preferred that the polymer (A) further has a side chain which includes a phenolic hydroxyl group. The side chain is preferably a structural unit (third structural unit; hereinafter, may be also referred to as “structural unit (III)”) which includes a phenolic hydroxyl group.

In a case of KrF exposure, EUV exposure, or electron beam exposure, due to the polymer (A) having the structural unit (III), sensitivity of the radiation-sensitive composition can be more enhanced. Therefore, in the case in which the polymer (A) has the structural unit (III), the radiation-sensitive composition can be suitably used as a radiation-sensitive composition for KrF exposure, for EUV exposure, or for electron beam exposure.

Examples of the structural unit (III) include a structural unit represented by the following formula (III-1) (hereinafter, structural unit (III-1)), and the like.

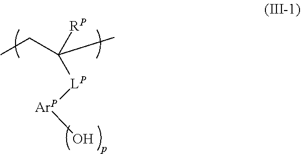

In the above formula (III-1), R^{P} represents a hydrogen atom, a fluorine atom, a methyl group, or a trifluoromethyl group; L^{P} represents a single bond, —COO—, —O—, or —CONH—; Ar^{P} represents a group obtained by removing (p+1) hydrogen atoms from a substituted or unsubstituted aromatic hydrocarbon ring structure having 6 to 30 ring atoms; and p is an integer of 1 to 3.

R^{P} represents preferably a hydrogen atom or a methyl group, in light of a degree of copolymerization of the monomer that gives the structural unit (III-1).

L^{P} represents preferably a single bond or —COO—, and more preferably a single bond. In the case in which L^{P} represents a single bond, CDU of the radiation-sensitive composition can be more improved.

Examples of the aromatic hydrocarbon ring structure having 6 to 30 ring atoms that gives Ar^{P} include, among the examples of the aromatic ring structure having 5 to 30 ring atoms that gives Ar¹ in the above formula (1-1), the ring structures similar to those exemplified as the aromatic hydrocarbon ring structure having 6 to 30 ring atoms, and the like. Of these, a benzene structure or a naphthalene structure is preferred, and a benzene structure is more preferred.

A part or all of hydrogen atoms in the aromatic hydrocarbon ring structure may be substituted with a substituent. Examples of the substituent include groups similar to those exemplified as the substituent which may be included in the above-described ring structure that gives Ar¹, and the like.
p is preferably 1 or 2. In the case in which p is 1, CDU of the radiation-sensitive composition can be more improved. In the case in which p is 2, the sensitivity of the radiation-sensitive composition can be more improved.

Moreover, in the case in which p is 1 and L^{P} represents —COO—, the hydroxy group is preferably bonded to the carbon atom which is adjacent to the carbon atom bonding to L^{P}, among the carbon atoms constituting Ar^{P}. In the case in which p is no less than 2 and L^{P} represents —COO—, at least one hydroxy group is preferably bonded to the carbon atom which is adjacent to the carbon atom bonding to L^{P}, among the carbon atoms constituting Ar^{P}. In other words, at least one hydroxy group and L^{P} preferably bond to an ortho position each other in Ar^{P}. In this case, generation of defects in the resist pattern to be formed with the radiation-sensitive composition can be inhibited.

Examples of the structural unit (III-1) include structural units represented by the following formulae (III-1-1) to (III-1-20) (hereinafter, may be also referred to as “structural units (III-1-1) to (III-1-20)”), and the like.

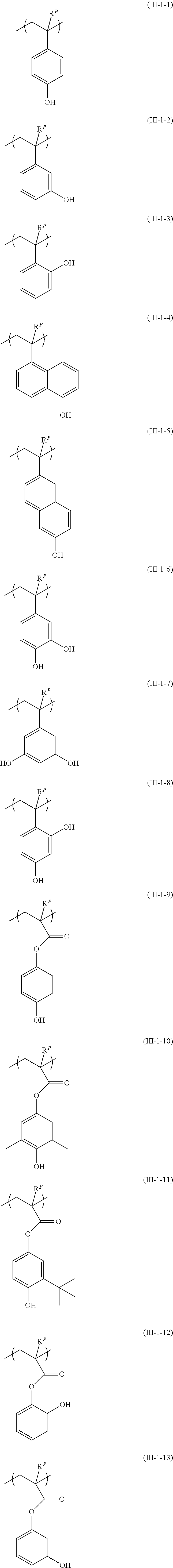

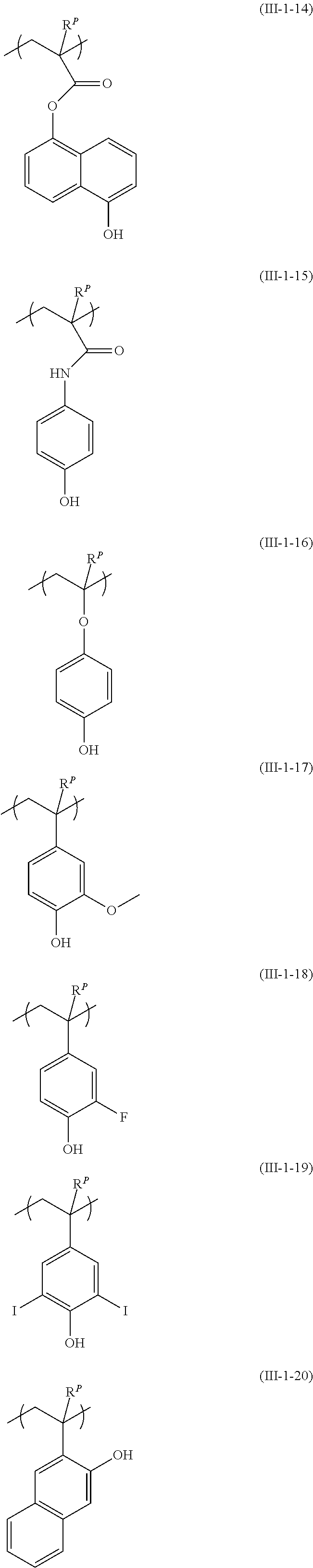

In the above formulae (III-1-1) to (III-1-20), R^{P} is as defined in the above formula (III-1).

In the case in which the polymer (A) has the structural unit (III), the lower limit of a proportion of the structural unit (III) contained in the polymer (A) with respect to the total structural units constituting the polymer (A) is preferably 10 mol %, more preferably 15 mol %, still more preferably 20 mol %, and particularly preferably 25 mol %. The upper limit of the proportion is preferably 60 mol %, more preferably 50 mol %, still more preferably 45 mol %, and particularly preferably 40 mol %.

As the monomer that gives a structural unit (III), a monomer obtained by substituting with an acetyl group, etc., a hydrogen atom of a phenolic hydroxyl group (—OH) of 4-acetoxystyrene, 3,5-diacetoxystyrene, or the like can be also used. In this case, for example, the polymer (A) having the structural unit (III) can be synthesized by after polymerizing the monomer, a resultant polymerization reaction product is subjected to a hydrolysis reaction in the presence of a base such as an amine.

### Other Structural Unit

The other structural unit is a structural unit other than the structural units (I) to (III). The other structural unit is exemplified by: a structural unit (“hereinafter, may be also referred to as “structural unit (IV)) which includes a lactone structure, a cyclic carbonate structure, a sultone structure, or a combination thereof; a structural unit (hereinafter, may be also referred to as “structural unit (V)”) which includes an alcoholic hydroxyl group; and the like.

### Structural Unit (IV)

The structural unit (IV) is a structural unit which includes a lactone structure, a cyclic carbonate structure, a sultone structure, or a combination thereof. When the polymer (A) further has the structural unit (IV), the adhesiveness to the substrate can be improved.

Examples of the structural unit (IV) include structural units represented by the following formulae, and the like.

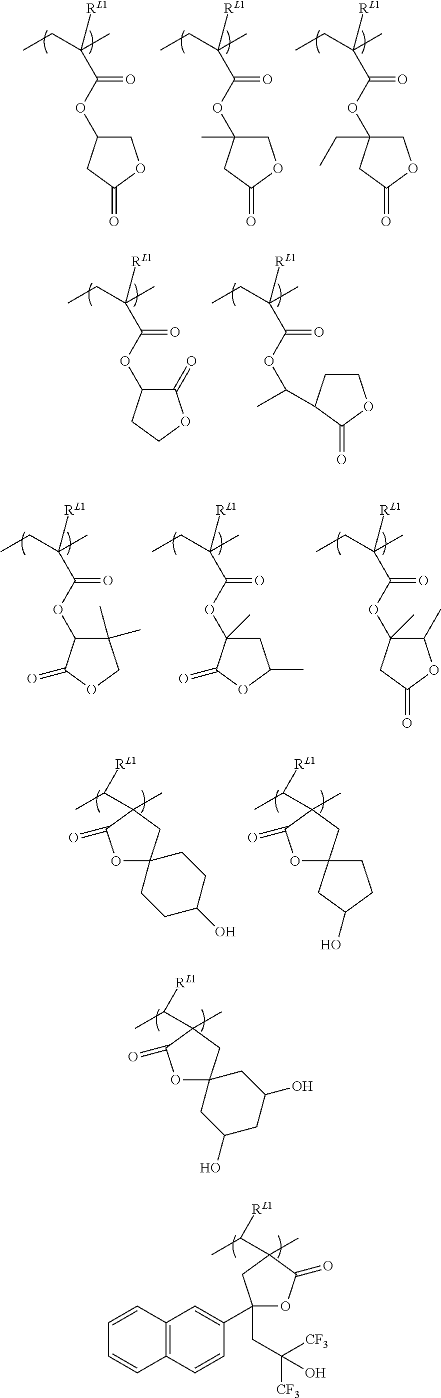

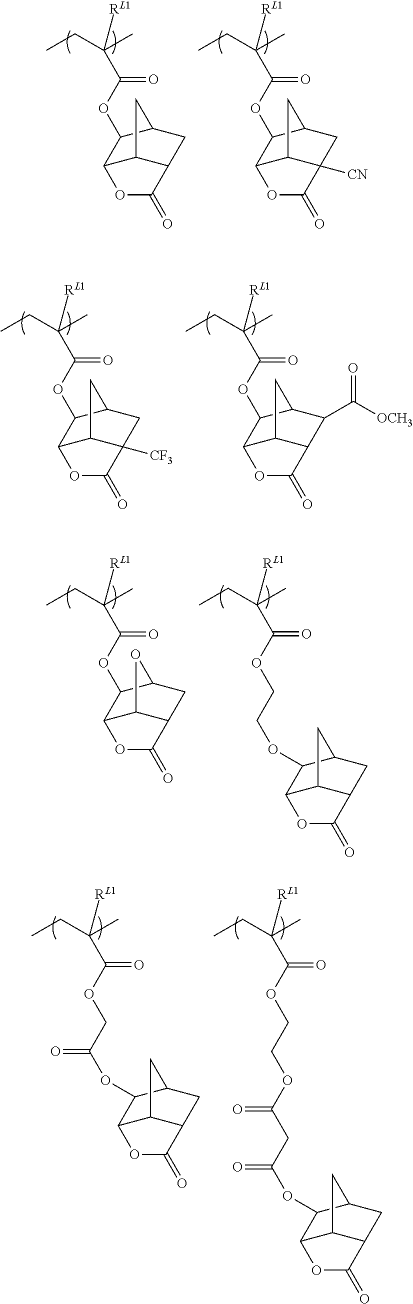

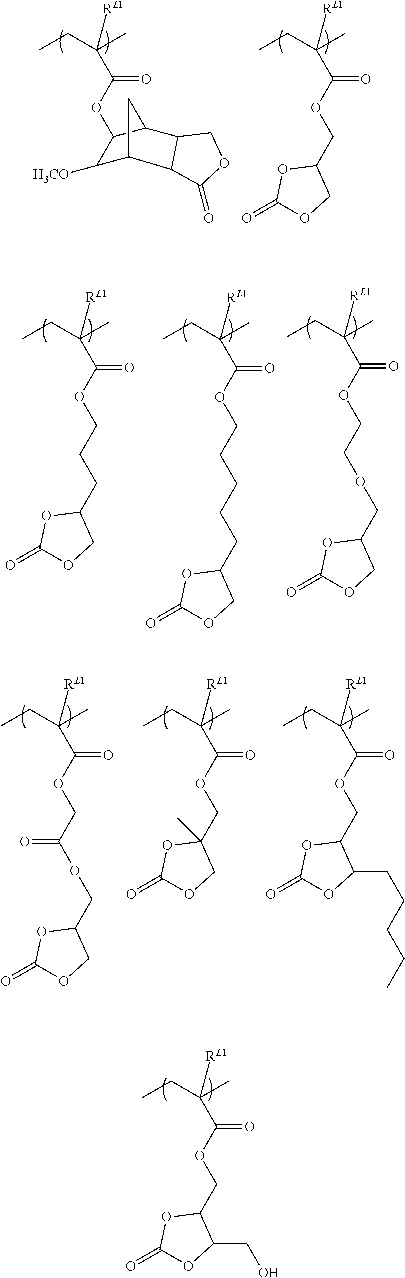

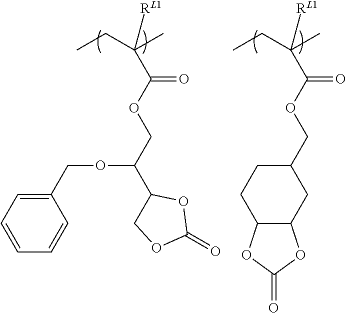

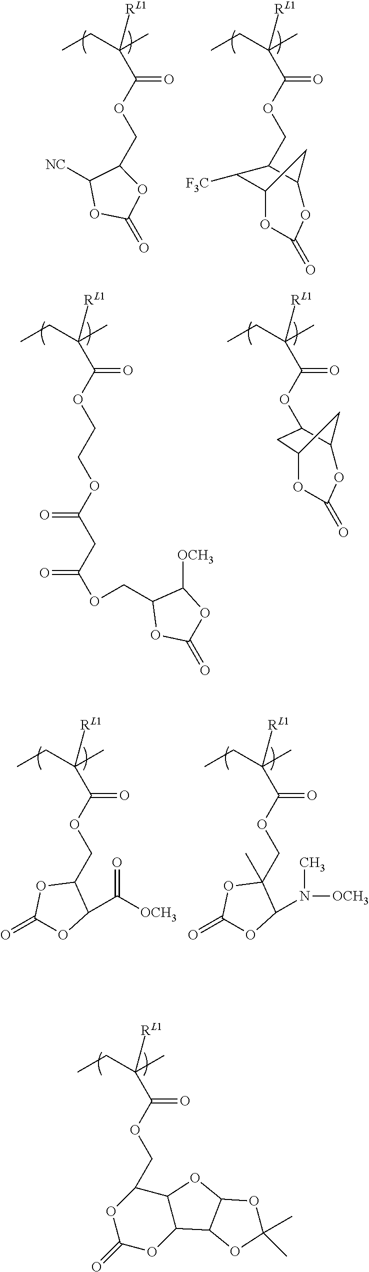

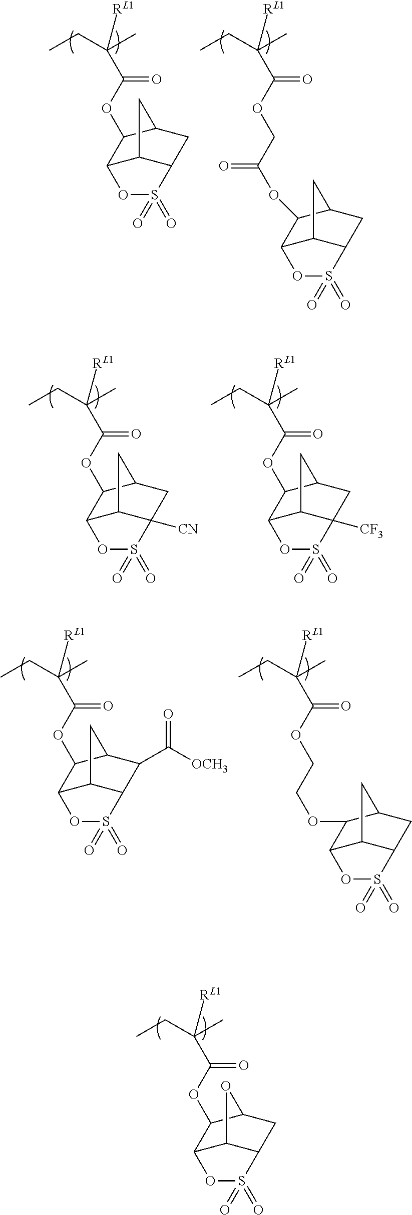

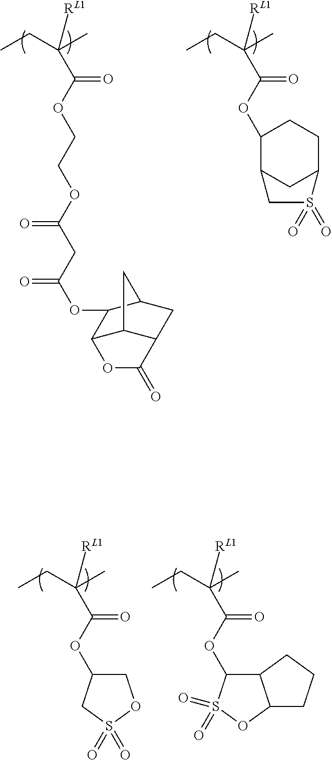

In the above formulae, R^{L1} represents a hydrogen atom, a fluorine atom, a methyl group, or a trifluoromethyl group.

The structural unit (IV) is preferably a structural unit which includes a lactone structure.

In the case in which the polymer (A) has the structural unit (IV), the lower limit of a proportion of the structural unit (IV) is, with respect to the total structural units constituting the polymer (A), preferably 5 mol %, and more preferably 10 mol %. The upper limit of the proportion is preferably 35 mol %, and more preferably 25 mol %.

### Structural Unit (V)

The structural unit (V) is a structural unit which includes an alcoholic hydroxyl group (wherein, those falling under the structural unit (IV) is excluded). When the polymer (A) further has the structural unit (V), solubility in a developer solution can be more appropriately adjusted.

Examples of the structural unit (V) include structural units represented by the following formulae, and the like.

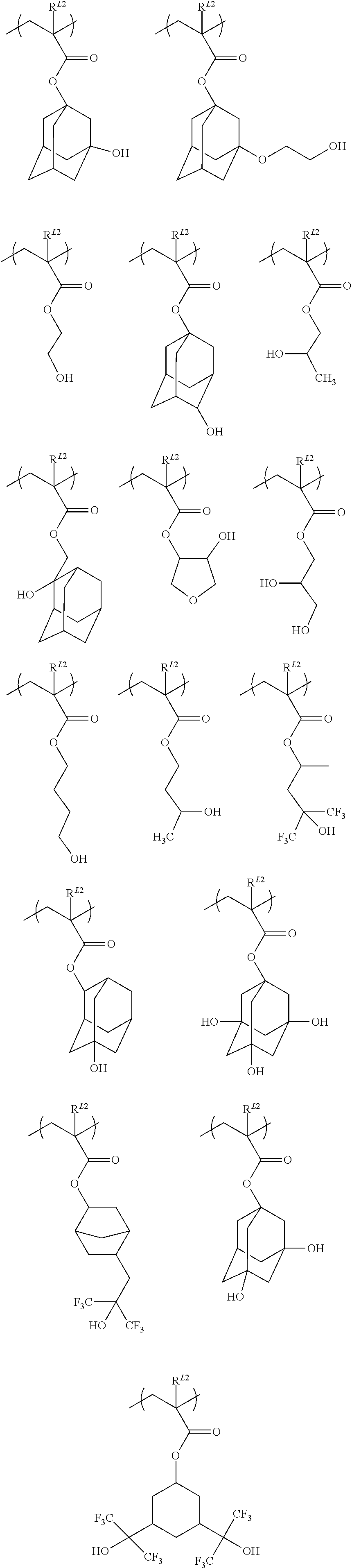

In the above formulae, R^{L2} represents a hydrogen atom, a fluorine atom, a methyl group, or a trifluoromethyl group.

In the case in which the polymer (A) has the structural unit (V), the lower limit of a proportion of structural unit (V) is, with respect to the total structural units constituting the polymer (A), preferably 5 mol %, and more preferably 10 mol %. The upper limit of the proportion is preferably 35 mol %, and more preferably 25 mol %.

### (B) Acid Generating Agent

The acid generating agent (B) is a substance that generates an acid upon an exposure (wherein, the polymer (A) is excluded), and a suitable molecular weight is no greater than 2,500, and particularly no greater than 1,500. Examples of the radioactive ray to be used for the exposure include radioactive rays similar to those exemplified as a radioactive ray in the exposure step of the method of forming a resist pattern described later, and the like. The acid-labile group included in the polymer (A), etc., is dissociated by an acid generated upon the exposure to generate a carboxy group or a phenolic hydroxyl group, whereby a resist pattern can be formed owing to the difference in solubility of the resist film in the developer solution caused between light-exposed region and light-unexposed regions. It is to be noted that as the acid generating agent (B), a polymer (P) that differs from the polymer (A) can also be used. The radiation-sensitive composition of the present disclosure has a radiation-sensitive onium cation structure in the polymer (A); however, the polymer (P) does not have the radiation-sensitive onium cation structure.

Examples of the acid generated from the acid generating agent (B) include sulfonic acid, carboxylic acid, imidic acid, and the like.

The acid generating agent (B) is exemplified by an onium salt compound, an N-sulfonyloxyimide compound, a sulfonimide compound, a halogen-containing compound, a diazoketone compound, and the like.

Examples of the onium salt compound include a sulfonium salt, a tetrahydrothiophenium salt, an iodonium salt, a phosphonium salt, a diazonium salt, a pyridinium salt, and the like.

Specific examples of the acid generating agent (B) include compounds disclosed in paragraph Nos. [0080] to[0113] of Japanese Unexamined Patent Application, Publication No. 2009-134088, and the like.

The acid generating agent (B) is preferably the onium salt compound, and more preferably an onium salt compound consisting of a radiation-sensitive onium cation and an organic acid anion.

Examples of the radiation-sensitive onium cation include monovalent cations (hereinafter, may be also referred to as “cations (r-a) to (r-b)”) represented by the following formulae (r-a) to (r-b), and the like.

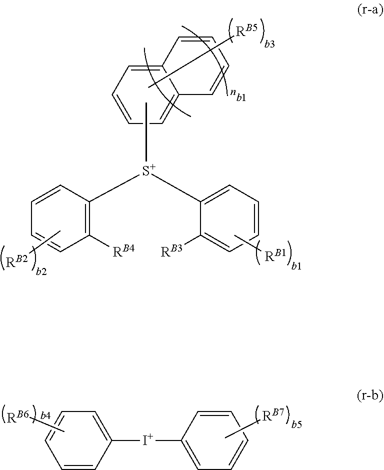

In the above formula (r-a), b1 is an integer of 0 to 4, wherein in the case in which b1 is 1, R^{B1} represents a halogen atom, a hydroxy group, a nitro group, or a monovalent organic group having 1 to 20 carbon atoms, and in the case in which b1 is no less than 2, a plurality of R^{B1}s are identical to or different from each other, and represent a halogen atom, a hydroxy group, a nitro group, or a monovalent organic group having 1 to 20 carbon atoms, or the R^{B1}s taken together represent a ring structure having 4 to 20 ring atoms together with the carbon chain to which R^{B1}s bond; b2 is an integer of 0 to 4, wherein in the case in which b2 is 1, R^{B2} represents a halogen atom, a hydroxy group, a nitro group, or a monovalent organic group having 1 to 20 carbon atoms, and in the case in which b2 is no less than 2, a plurality of R^{B2}S are identical to or different from each other, and represent a halogen atom, a hydroxy group, a nitro group, or a monovalent organic group having 1 to 20 carbon atoms, or R^{B2}S taken together represent a ring structure having 4 to 20 ring atoms together with the carbon chain to which R^{B2}s bond; R^{B3} and R^{B4} each independently represent a hydrogen atom, a halogen atom, a hydroxy group, a nitro group, or a monovalent organic group having 1 to 20 carbon atoms, or R^{B3} and R^{B4} taken together represent a single bond; b3 is an integer of 0 to 11, wherein in the case in which b3 is 1, R^{B5} represents a halogen atom, a hydroxy group, a nitro group, or a monovalent organic group having 1 to 20 carbon atoms, and in the case in which b3 is no less than 2, a plurality of R^{B5}s are identical to or different from each other, and represent a halogen atom, a hydroxy group, a nitro group, or a monovalent organic group having 1 to 20 carbon atoms, or R^{B5}S taken together represent a ring structure having 4 to 20 ring atoms together with the carbon chain to which R^{B5}s bond; and n_{b1} is an integer of 0 to 3.

In the above formula (r-b), b4 is an integer of 0 to 5, wherein in the case in which b4 is 1, R^{B6} represents a halogen atom, a hydroxy group, a nitro group, or a monovalent organic group having 1 to 20 carbon atoms, and in the case in which b4 is no less than 2, a plurality of R^{B6}s are identical to or different from each other, and represent a halogen atom, a hydroxy group, a nitro group, or a monovalent organic group having 1 to 20 carbon atoms, or R^{B6}s taken together represent a ring structure having 4 to 20 ring atoms together with the carbon chain to which R^{B6}s bond; and b5 is an integer of 0 to 5, wherein in the case in which b5 is 1, R^{B7} represents a halogen atom, a hydroxy group, a nitro group, or a monovalent organic group having 1 to 20 carbon atoms, and in the case in which b5 is no less than 2, a plurality of R^{B7}s are identical to or different from each other, and represent a halogen atom, a hydroxy group, a nitro group, or a monovalent organic group having 1 to 20 carbon atoms, or R^{B6}s taken together represent a ring structure having 4 to 20 ring atoms together with the carbon chain to which R^{B6}s bond.

The “organic group” as referred to herein means a group which includes at least one carbon atom.

The monovalent organic group having 1 to 20 carbon atoms which may be represented by each of R^{B1}, R^{B2}, R^{B3}, R^{B4}, R^{B5} and R^{B6} is exemplified by: a monovalent hydrocarbon group having 1 to 20 carbon atoms; a group (α) including a divalent hetero atom-containing group between two adjacent carbon atoms of the monovalent hydrocarbon group; a group (β) obtained by substituting with a monovalent hetero atom-containing group, a part or all of hydrogen atoms included in the monovalent hydrocarbon group or the group (α); a group (γ) in which the monovalent hydrocarbon group, the group (α), or the group (β) is combined with a divalent hetero atom-containing group; and the like.

Examples of the monovalent hydrocarbon group having 1 to 20 carbon atoms include groups similar to the groups exemplified as the monovalent hydrocarbon group having 1 to 20 carbon atoms which may be represented by R^{B}, R^{C}, R^{U}, R^{V}, or R^{W} in the above formulae (b-2) to (b-3), and the like.

The hetero atom that may constitute the monovalent or divalent hetero atom-containing group is exemplified by an oxygen atom, a nitrogen atom, a sulfur atom, a phosphorus atom, a silicon atom, a halogen atom, and the like. Examples of the halogen atom include a fluorine atom, a chlorine atom, a bromine atom, and an iodine atom.

Examples of the divalent hetero atom-containing group include —O—, —CO—, —S—, —CS—, —NR′—, groups in which at least two of the aforementioned groups are combined (for example, —COO—, —CONR′—, etc.), and the like, wherein R′ represents a hydrogen atom or a monovalent hydrocarbon group having 1 to 10 carbon atoms.

R^{B1}, R^{B2}, R^{B5}, R^{B6}, and R^{B7} represent preferably a halogen atom or a group obtained by substituting a part or all of hydrogen atoms included in a monovalent hydrocarbon group having 1 to 20 carbon atoms, with a monovalent halogen atom. The halogen atom in this case is preferably a fluorine atom or an iodine atom. In this case, a favorable balance of the sensitivity and CDU of the radiation-sensitive composition can be contemplated.

R^{B3} and R^{B4} represent preferably a hydrogen atom. or taken together represent a single bond.

b1, b2 and b3 are preferably 0 to 3. n_{b1} is preferably 0 or 1.

b4 and b5 are each preferably 0 or 1.

Of the cations (r-a) to (r-b) described above, a monovalent radiation-sensitive sulfonium cation (hereinafter, may be also referred to as “cation (P)”) which includes an aromatic ring structure having at least one selected from the group consisting of a fluorine atom, a fluorine atom-containing group, and an iodine atom is preferred. The cation (r-a) falling under the cation (P) is exemplified by a cation in which b1 is an integer of 1 to 3, and at least one R^{B1} is at least one group selected from the group consisting of a fluorine atom, a fluorine atom-containing group, and an iodine atom. Of these, a cation in which: b1 and b2 are each independently an integer of 1 to 3; at least one R^{B1} represents a fluorine atom or an iodine atom; and at least one R^{B2} represents a fluorine atom or an iodine atom is preferred. Furthermore, the cation (r-b) falling under the cation (P) is exemplified by a cation in which b4 is an integer of 1 to 5, and at least one R^{B6} represents a fluorine atom or an iodine atom. Of these, a cation in which: b4 and b5 are each independently an integer of 1 to 5; at least one R^{B6} represents a fluorine atom or an iodine atom; and at least one R^{B7} represents a fluorine atom or an iodine atom is preferred.

Examples of the organic acid anion include a sulfonate anion, a carboxylate anion, an imidate anion, and the like].

Of these, the acid generating agent (B) is preferably an onium salt compound (hereinafter, may be also referred to as “compound (Z)”) having the cation (P) and a monovalent organic acid anion (hereinafter, may be also referred to as “anion (Q)”).

Examples of the cation (P) include cations (hereinafter, may be also referred to as “cations (P-1-1) to (P-1-12)”) represented by the following formulae (2-1-1) to (2-1-12), and the like. Examples of the radiation-sensitive onium cation not corresponding to the cation (P) include a triphenylsulfonium cation and a diphenyliodonium cation.

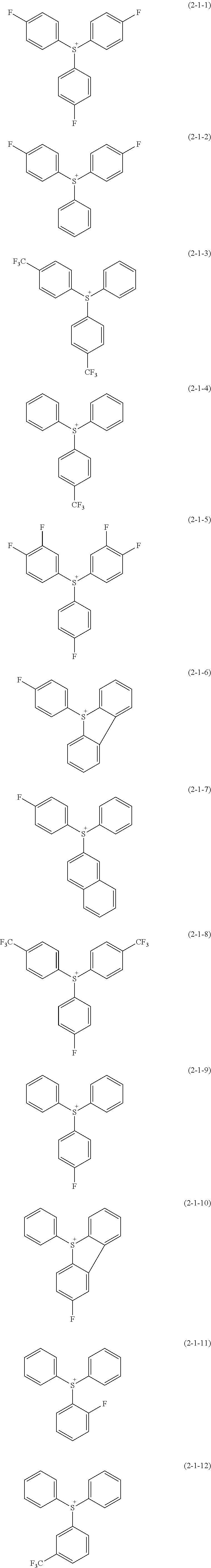

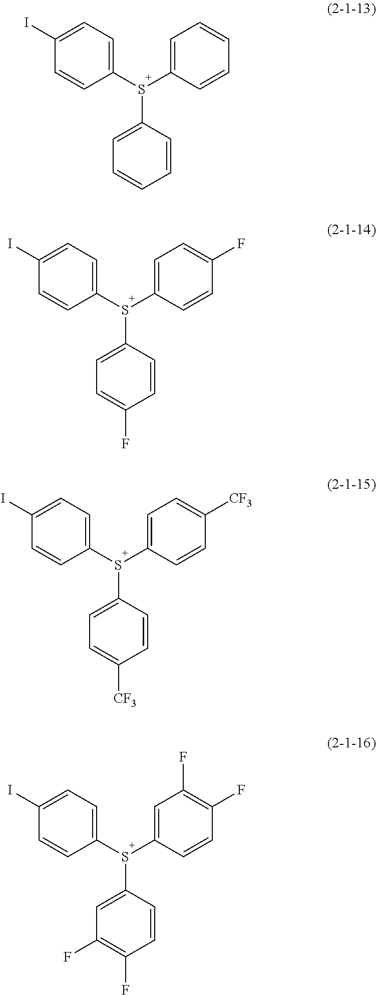

### Anion (Q)

The anion (Q) is a monovalent organic acid anion. The anion (Q) includes a monovalent anion group. Examples of the monovalent anion group include a sulfonate anion group, a carboxylate anion group, an imidate anion group, and the like. Of these, a sulfonate anion group, or a carboxylate anion group is preferred.

Of the anions (Q), an anion (hereinafter, may be also referred to as “anion (Q-1)”) having a sulfonate anion group as the monovalent anion group is described below.

### Anion (Q-1)

An anion moiety (Q-1) is not particularly limited as long as it is a sulfonate anion to be used as an anion in a radiation-sensitive acid generating agent of an onium salt type, and is exemplified by a sulfonate anion represented by the following formula (4-1).

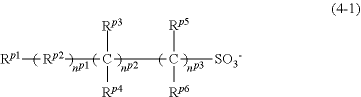

In the above formula (4-1), R^{p1} represents a monovalent group which includes a ring structure having five or more ring atoms; R^{p2} represents a divalent linking group; R^{p3} and R^{p4} each independently represent a hydrogen atom, a fluorine atom, a monovalent hydrocarbon group having 1 to 20 carbon atoms, or a monovalent fluorinated hydrocarbon group having 1 to 20 carbon atoms; R^{p5} and R^{p6} each independently represent a fluorine atom or a monovalent fluorinated hydrocarbon group having 1 to 20 carbon atoms; n^{p1} is an integer of 0 to 10; n^{p2} is an integer of 0 to 10; and n^{p3} is an integer of 0 to 10, wherein a sum of n^{p1}, n^{p2}, and n^{p3} is no less than 1 and no greater than 30, and wherein in a case in which n^{p1} is no less than 2, a plurality of R^{p2}s are identical to or different from each other, in a case in which n^{p2} is no less than 2, a plurality of R^{p3}s are identical to or different from each other, a plurality of R^{p4}s are identical to or different from each other, and in a case in which n^{p3} is no less than 2, a plurality of R^{p5}s are identical to or different from each other, a plurality of R^{p6}s are identical to or different from each other.

The ring structure having five or more ring atoms is exemplified by an aliphatic hydrocarbon ring structure having five or more ring atoms, an aliphatic heterocyclic structure having five or more ring atoms, an aromatic hydrocarbon ring structure having six or more ring atoms, an aromatic heterocyclic structure having five or more ring atoms or a combination of the same.

Examples of the aliphatic hydrocarbon ring structure having five or more ring atoms include: monocyclic saturated alicyclic structures such as a cyclopentane structure, a cyclohexane structure, a cycloheptane structure, a cyclooctane structure, a cyclononane structure, a cyclodecane structure, and a cyclododecane structure; monocyclic unsaturated alicyclic structures such as a cyclopentene structure, a cyclohexene structure, a cycloheptene structure, a cyclooctene structure, and a cyclodecene structure; polycyclic saturated alicyclic structures such as a norbornane structure, an adamantane structure, a tricyclodecane structure, a tetracyclododecane structure, and a steroid structure; polycyclic unsaturated alicyclic structures such as a norbornene structure and a tricyclodecene structure; and the like. The “steroid structure” as referred to herein means a structure having, as a basic skeleton, a skeleton (sterane skeleton) provided by condensation of three 6-membered rings and one 5-membered ring. Of the examples of the ring structure, the steroid structure is preferred.

Examples of the aliphatic heterocyclic structure having five or more ring atoms include: lactone structures such as a hexanolactone structure and a norbornanelactone structure; sultone structures such as a hexanosultone structure and a norbornanesultone structure; oxygen atom-containing heterocyclic structures such as a dioxolane structure, an oxacycloheptane structure, and an oxanorbornane structure; nitrogen atom-containing heterocyclic structures such as an azacyclohexane structure and a diazabicyclooctane structure; sulfur atom-containing heterocyclic structures such as a thiacyclohexane structure and a thianorbornane structure; and the like.

Examples of the aromatic hydrocarbon ring structure having six or more ring atoms include: a benzene structure; condensed polycyclic aromatic hydrocarbon ring structures such as a naphthalene structure, an anthracene structure, a fluorene structure, a biphenylene structure, a phenanthrene structure, and a pyrene structure; ring-assembled aromatic hydrocarbon ring structures such as a biphenyl structure, a terphenyl structure, a binaphthalene structure, and a phenylnaphthalene structure; a 9,10-ethanoanthracene structure; a triptycenestructure; and the like. Of these, a benzene structure and a 9,10-ethanoanthracene structure are preferred.

Examples of the aromatic heterocyclic structure include furan structure having five or more ring atoms include: oxygen atom-containing heterocyclic structures such as a pyran structure, a benzofuran structure, and a benzopyran structure; nitrogen atom-containing heterocyclic structures such as a pyridine structure, a pyrimidine structure, and an indole structure; sulfur atom-containing heterocyclic structures such as a thiophene structure; and the like.

In the ring structure described above, a part or all of hydrogen atoms bonding to an atom constituting the ring structure may be substituted with a substituent. Examples of the substituent include halogen atoms such as a fluorine atom and an iodine atom, a hydroxy group, a carboxy group, a cyano group, a nitro group, an alkyl group, an alkoxy group, an alkoxycarbonyl group, an alkoxycarbonyloxy group, an acyl group, an acyloxy group, an oxo group (═O), and the like.

The lower limit of the number of ring atoms of the ring structure described above is preferably 6, more preferably 8, still more preferably 9, and particularly preferably 10. The upper limit of the number of ring atoms is preferably 25.

R^{p1} represents preferably a monovalent group which includes the aliphatic hydrocarbon ring structure having five or more ring atoms, a monovalent group which includes the aliphatic heterocyclic structure having five or more ring atoms, or a monovalent group which includes the aromatic hydrocarbon ring structure having six or more ring atoms. Of these, a monovalent group which includes the aromatic hydrocarbon ring structure having six or more ring atoms and having one to four iodine atom(s) as the substituent(s) is preferred.

Examples of the divalent linking group represented by R^{p2} include a carbonyl group, an ether group, a carbonyloxy group, a sulfide group, a thiocarbonyl group, a sulfonyl group, a divalent hydrocarbon group, a group obtained by combining the same, and the like. The carbon atom constituting the divalent hydrocarbon group may be replaced with a carbonyl group or an ether group.

The monovalent hydrocarbon group having 1 to 20 carbon atoms which may be represented by each of R^{p1} and R^{p4} is exemplified by an alkyl group having 1 to 20 carbon atoms, and the like. The monovalent fluorinated hydrocarbon group having 1 to 20 carbon atoms which may be represented by each of R^{p3} and R^{p4} is exemplified by a fluorinated alkyl group having 1 to 20 carbon atoms, and the like. R^{p3} and R^{p4} each represent preferably a hydrogen atom, a fluorine atom, or a fluorinated alkyl group, more preferably a hydrogen atom, a fluorine atom, or a perfluoroalkyl group, and still more preferably a hydrogen atom, a fluorine atom, or a trifluoromethyl group.

The monovalent fluorinated hydrocarbon group having 1 to 20 carbon atoms which may be represented by each of R^{p1} and R^{p6} is exemplified by a fluorinated alkyl group having 1 to 20 carbon atoms, and the like. R^{p5} and R^{p6} each represent preferably a fluorine atom or a fluorinated alkyl group, more preferably a fluorine atom or a perfluoroalkyl group, still more preferably a fluorine atom or a trifluoromethyl group, and particularly preferably a fluorine atom.
n^{p1} is preferably 0 to 5, more preferably 0 to 2, and still more preferably 0 or 1.
n^{p2} is preferably 0 to 5, more preferably 0 to 2, and still more preferably 0 or 1.

The lower limit of n^{p3} is preferably 1, and more preferably 2. When n^{p3} is no less than 1, strength of the acid can be enhanced. The upper limit of n^{p3} is preferably 4, more preferably 3, and still more preferably 2.

The lower limit of the sum of n^{p1}, n^{p2}, and n^{p3} is preferably 2, and more preferably 4. The upper limit of the sum of n^{p1}, n^{p2}, and n^{p3} is preferably 20, and more preferably 10.

As the anion (Q-1), sulfonate anions represented by the following formulae (4-1-1) to (4-1-16) are preferred.

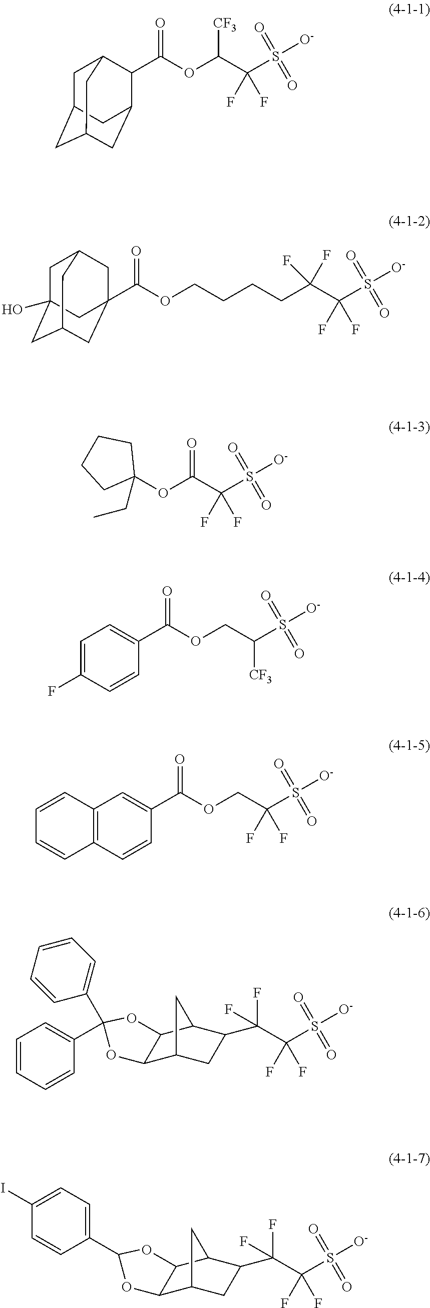

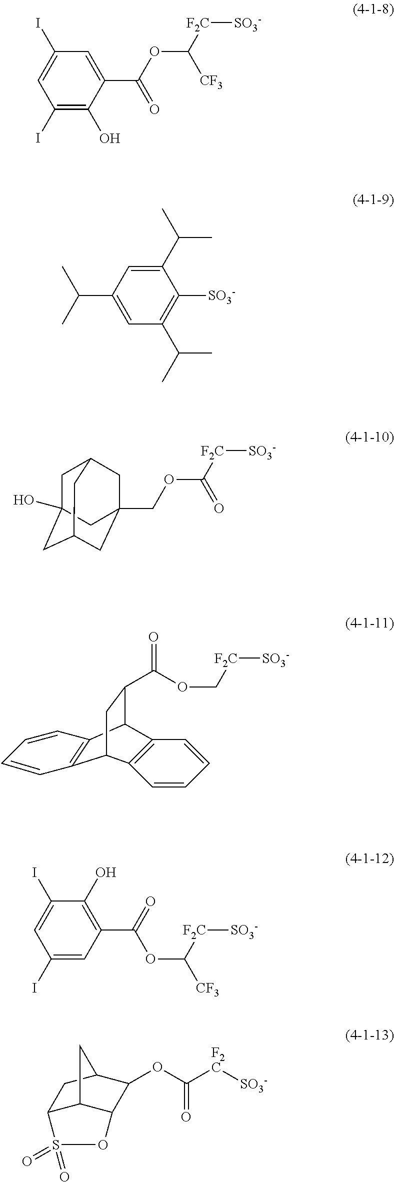

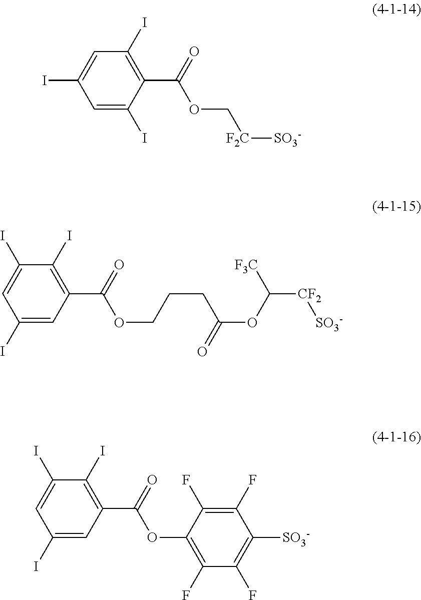

Of the anions (Q), for the anion having a carboxylate anion group as the monovalent anion group, an anion structure obtained by replacing the sulfonate anion in the above formula (4-1) with a carboxylate anion is applicable.

The lower limit of a content of the acid generating agent (B) in the radiation-sensitive composition is, with respect to 100 parts by mass of the polymer (A), preferably 1 part by mass, more preferably 5 parts by mass, and still more preferably 10 parts by mass. The upper limit of the content is preferably 50 parts by mass, more preferably 40 parts by mass, and still more preferably 30 parts by mass.

### (C) Acid Diffusion Control Agent

The acid diffusion control agent (C) is capable of controlling a diffusion phenomenon in the resist film of the acid generated from the polymer (A), the acid generating agent (B), and the like upon exposure, thereby serving to inhibit unwanted chemical reactions in light-unexposed regions. The acid diffusion control agent (C) is exemplified by a compound (hereinafter, may be also referred to as “photodegradable base”) having a monovalent radiation-sensitive onium cation and a monovalent organic acid anion (wherein, the polymer (A) is excluded). Although the photodegradable base may be considered to fall under the category of the acid generating agent in a broad sense due to being capable of generating an acid upon exposure, the photodegradable base does not allow for dissociation of the acid-labile group upon exposure under conditions which permit dissociation of the acid-labile group in the polymer (A) by the acid generated from the polymer (A) and/or the acid generating agent (B). The photodegradable base has a molecular weight of preferably no greater than 2,500, and more preferably no greater than 1,500. It is to be noted that as the acid diffusion control agent (C), a having a repeating unit that can serve as described above can be also used.

Examples of the monovalent radiation-sensitive onium cation in the photodegradable base include onium cations similar to those exemplified as the cation of the acid generating agent (B), and the like. Of these, a monovalent radiation-sensitive sulfonium cation (cation (P)) which includes an aromatic ring structure having at least one selected from the group consisting of a fluorine atom, a fluorine atom-containing group, and an iodine atom is preferred.

The monovalent organic acid anion in the photodegradable base includes a monovalent anion group. Examples of the monovalent anion group include a carboxylate anion group, an imidate anion group, and the like. Of these, a carboxylate anion group is preferred.

Of the anions (Q), an anion (hereinafter, may be also referred to as “anion (Q-2)”) having a carboxylate anion group as the monovalent anion group is described below.

The anion (Q-2) is not particularly limited as long as it can be used as an anion in a photodegradable base that generates a weak acid through photosensitization upon exposure. In particular, a carboxylate anion which includes an aromatic ring structure having one to three iodo group(s) introduced by substitution of one to three hydrogen atom(s) is preferred, and a carboxylate anion which includes an aromatic ring structure having two to three iodo groups introduced by substitution of two to three hydrogen atoms is more preferred.

The anion moiety (Q-2) is preferably a carboxylate anion represented by each of the following formulae (4-2-1) to (4-2-12).

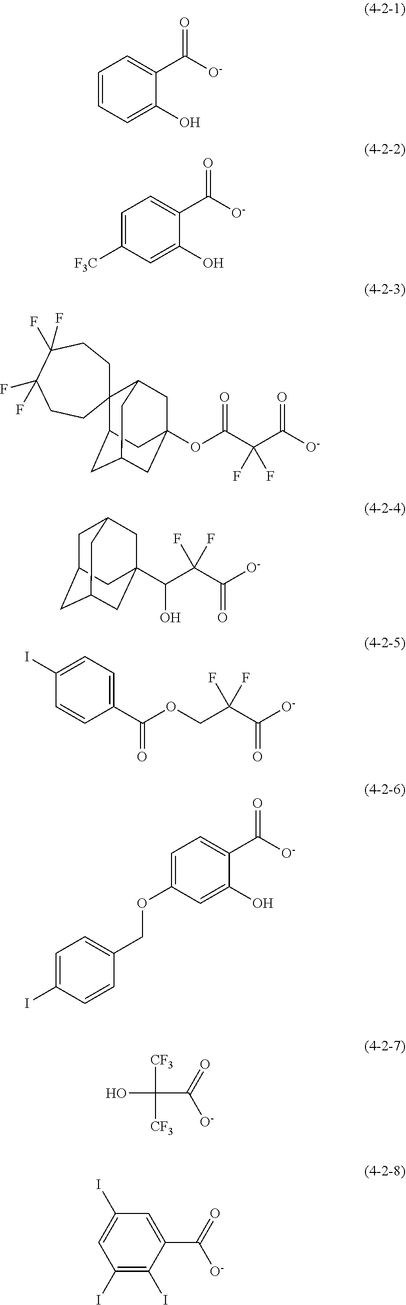

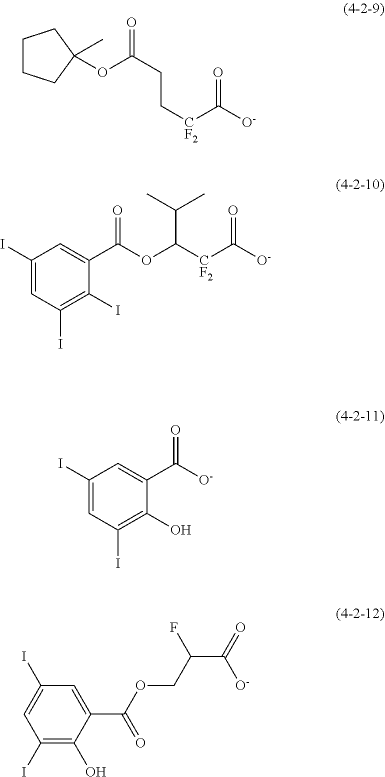 [SMILES: CC1(OC(=O)CCC(F)(F)C(=O)[O-])CCCC1 ; CC(C)C(OC(=O)c1cc(I)cc(I)c1I)C(F)(F)C(=O)[O-] ; O=C([O-])c1cc(I)cc(I)c1O ; O=C(OCC(F)C(=O)[O-])c1cc(I)cc(I)c1O]

The photodegradable base which can be used is exemplified by a compound obtained by combining a monovalent radiation-sensitive onium cation and the anion moiety (Q-2) ad libitum.

As the acid diffusion control agent (C), a nitrogen atom-containing compound is also applicable as a compound other than the photodegradable base. Examples of the nitrogen atom-containing compound include: amine compounds such as tripentylamine and trioctylamine; amide group-containing compounds such as formamide and N,N-dimethylacetamide; urea compounds such as urea and 1,1-dimethylurea; nitrogen-containing heterocyclic compounds such as pyridine, N-(undecylcarbonyloxyethyl)morpholine, and N-t-pentyloxycarbonyl-4-hydroxypiperidine; and the like.

In the case in which the radiation-sensitive composition contains the acid diffusion control agent (C), the lower limit of a content of the acid diffusion control agent (C) in the radiation-sensitive composition is, with respect to 100 parts by mass of the polymer (A) contained in the radiation-sensitive composition, preferably 1 part by mass, more preferably 3 parts by mass, and still more preferably 5 parts by mass. The upper limit of the content is preferably 30 parts by mass, more preferably 20 parts by mass, and still more preferably 15 parts by mass.

The lower limit of a content of the acid diffusion control agent (C) in the radiation-sensitive composition is, with respect to the acid generating agent (B) accounting for 100 mol %, preferably 1 mol %, more preferably 5 mol %, and still more preferably 10 mol %. The upper limit of the content is preferably 100 mol %, more preferably 50 mol %, and still more preferably 30 mol %.

Further, the lower limit of a content of the acid diffusion control agent (C) in the radiation-sensitive composition is, with respect to 100 parts by mass of the total of the polymer (A) and the acid generating agent (B), preferably 1 part by mass, more preferably 2 parts by mass, and still more preferably 5 parts by mass. The upper limit of the content is preferably 50 parts by mass, more preferably 40 parts by mass, and still more preferably 30 parts by mass.

### (D) Organic Solvent

The radiation-sensitive composition typically contains the organic solvent (D). The organic solvent (D) is not particularly limited as long as it is a solvent capable of dissolving or dispersing at least the polymer (A), and the acid generating agent (B), the acid diffusion control agent (C), and the polymer (F), as well as the other optional component(s) which may be contained as needed.

The organic solvent (D) is exemplified by an alcohol solvent, an ether solvent, a ketone solvent, an amide solvent, an ester solvent, a hydrocarbon solvent, and the like. The radiation-sensitive composition may contain one type, or two or more types of the organic solvent (D).

Examples of the alcohol solvent include: aliphatic monohydric alcohol solvents having 1 to 18 carbon atoms such as 4-methyl-2-pentanol, n-hexanol, and diacetonealcohol; alicyclic monohydric alcohol solvents having 3 to 18 carbon atoms such as cyclohexanol; polyhydric alcohol solvents having 2 to 18 carbon atoms such as 1,2-propylene glycol; polyhydric alcohol partial ether solvents having 3 to 19 carbon atoms such as propylene glycol monomethyl ether; and the like.

Examples of the ether solvent include: dialkyl ether solvents such as diethyl ether, dipropyl ether, dibutyl ether, dipentyl ether, diisoamyl ether, dihexyl ether, and diheptyl ether; cyclic ether solvents such as tetrahydrofuran and tetrahydropyran; aromatic ring-containing ether solvents such as diphenyl ether and anisole; and the like.

Examples of the ketone solvent include: chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone; cyclic ketone solvents such as cyclopentanone, cyclohexanone, cycloheptanone, cyclooctanone, and methylcyclohexanone; 2,4-pentanedione, acetonylacetone, and acetophenone; and the like.

Examples of the amide solvent include: cyclic amide solvents such as N,N′-dimethylimidazolidinone and N-methylpyrrolidone; chain amide solvents such as N-methylformamide, N,N-dimethylformamide, N,N-diethylformamide, acetamide, N-methylacetamide, N,N-dimethylacetamide, and N-methylpropionamide; and the like.

Examples of the ester solvent include: monocarboxylic acid ester solvents such as n-butyl acetate and ethyl lactate; lactone solvents such as γ-butyrolactone and valerolactone; polyhydric alcohol carboxylate solvents such as propylene glycol acetate; polyhydric alcohol partial ether carboxylate solvents such as propylene glycol monomethyl ether acetate; polyhydric carboxylic acid diester solvents such as diethyl oxalate; carbonate solvents such as dimethyl carbonate and diethyl carbonate; and the like.

Examples of the hydrocarbon solvent include: aliphatic hydrocarbon solvents having 5 to 12 carbon atoms such as n-pentane and n-hexane; aromatic hydrocarbon solvents having 6 to 16 carbon atoms such as toluene and xylene; and the like.

The organic solvent (D) is preferably the alcohol solvent, the ester solvent, or a combination of the same, more preferably the polyhydric alcohol partial ether solvent, the polyhydric alcohol partial ether carboxylate solvent having 3 to 19 carbon atoms, or a combination of the same, and still more preferably propylene glycol 1-monomethyl ether, propylene glycol monomethyl ether acetate, or a combination of the same.

In the case of the radiation-sensitive composition containing the organic solvent (D), the lower limit of a proportion of the organic solvent (D) with respect to total components contained in the radiation-sensitive composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

### (F) Polymer

The polymer (F) is a polymer being different from the polymer (A) and having a percentage content of fluorine atoms greater than that of the polymer (A). In general, a polymer being more hydrophobic than a polymer to be the base polymer tends to be localized in a resist film surface layer. Since the polymer (F) has a percentage content of fluorine atoms greater than that of the polymer (A), the polymer (F) tends to be localized in the resist film surface layer due to the characteristic resulting from hydrophobicity. As a result, in the case of the radiation-sensitive composition containing the polymer (F), a cross-sectional shape of a resist pattern to be formed is expected to be favorable. Moreover, in the case in which the radiation-sensitive composition contains the polymer (F), a cross-sectional shape of the resist pattern can be more improved.

The mode of incorporation of the fluorine atom in the polymer (F) is not particularly limited, and the fluorine atom may bond to any of the main chain and a side chain of the polymer (F). In a preferred mode of incorporation of the fluorine atom in the polymer (F), the polymer (F) has a structural unit (hereinafter, may be also referred to as “structural unit (F)”) which includes a fluorine atom. The polymer (F) may further have a structural unit other than the structural unit (F). The polymer (F) may have one, or two or more types of each structural unit.

In the case in which the radiation-sensitive composition contains the polymer (F), the lower limit of a content of the polymer (F) is, with respect to 100 parts by mass of the polymer (A), preferably 0.1 parts by mass, and more preferably 0.5 parts by mass. The upper limit of the content is preferably 10 parts by mass, and more preferably 5 parts by mass.

### Other Optional Component(s)

The other optional component(s) is/are exemplified by a surfactant and the like. The radiation-sensitive composition may contain one type, or two or more types each of the other optional component(s).

### Method of Forming Resist Pattern

The method of forming a resist pattern according to the other embodiment of the present disclosure includes: a step (hereinafter, may be also referred to as “applying step”) of applying a radiation-sensitive composition directly or indirectly on a substrate to form a resist film; a step (hereinafter, may be also referred to as “exposing step”) of exposing the resist film; and a step (hereinafter, may be also referred to as “developing step”) of developing the resist film exposed.

In the applying step, the radiation-sensitive composition of the one embodiment of the present disclosure is used as the radiation-sensitive composition. Therefore, the method of forming a resist pattern enables a resist pattern that is superior in CDU to be formed with favorable sensitivity.

Each step included in the method of forming a resist pattern will be described below.

### Applying Step

In this step, a radiation-sensitive composition is applied directly or indirectly on the substrate. Thus, the resist film is formed directly or indirectly on the substrate.

In this step, the radiation-sensitive composition of the one embodiment of the present disclosure, described above, is used as the radiation-sensitive composition.

The substrate is exemplified by a conventionally well-known substrate such as a silicon wafer and a wafer coated with silicon dioxide or aluminum, and the like. In addition, a mode of applying the radiation-sensitive composition indirectly on the substrate is exemplified by a mode of applying the radiation-sensitive composition on an antireflective film formed on the substrate, and the like. Examples of such an antireflective film include organic or inorganic antireflective films disclosed in Japanese Examined Patent Application, Publication No. H6-12452, Japanese Unexamined Patent Application, Publication No. S59-93448, etc., and the like.

An application procedure is exemplified by spin coating, cast coating, roll coating, and the like. After the application, prebaking (hereinafter, may be also referred to as “PB”) may be carried out as needed for evaporating the solvent remaining in the coating film. The lower limit of a PB temperature is preferably 60° C., and more preferably 80° C. The upper limit of the PB temperature is preferably 150° C., and more preferably 140° C. The lower limit of a PB time period is preferably 5 sec, and more preferably 10 sec. The upper limit of the PB time period is preferably 600 sec, and more preferably 300 sec. The lower limit of an average thickness of the resist film to be formed is preferably 10 nm, and more preferably 20 nm. The upper limit of the average thickness is preferably 1,000 nm, and more preferably 500 nm.

### Exposing Step

In this step, the resist film formed by the applying step is exposed. This exposure is carried out by irradiation with a radioactive ray through a photomask (as the case may be, through a liquid immersion medium such as water). The radioactive ray may be exemplified by, in accordance with the line widths of the pattern intended, and the like: electromagnetic waves such as a visible light ray, ultraviolet ray, a far ultraviolet ray, an extreme ultraviolet ray (EUV), an X-ray, and a 7-ray; charged particle rays such as an electron beam and an α-ray; and the like. Of these, far ultraviolet rays, EUV, or electron beams are preferred; an ArF excimer laser beam (wavelength: 193 nm), a KrF excimer laser beam (wavelength: 248 nm), EUV (wavelength: 13.5 mm), or an electron beam is more preferred; a KrF excimer laser beam, EUV, or an electron beam is still more preferred; and EUV or an electron beam is particularly preferred.

It is preferred that post exposure baking (hereinafter, may be also referred to as “PEB”) is carried out after the exposure, thereby promoting dissociation of the acid-labile group from the structural unit (I) in exposed portions of the resist film, by way of the action of the acid generated from the polymer (A) and the like upon the exposure. This PEB enables an increase in a difference in solubility of the resist film in a developer solution between the light-exposed regions and light-unexposed regions. The lower limit of a PEB temperature is preferably 50° C., and more preferably 80° C. The upper limit of the PEB temperature is preferably 180° C., and more preferably 130° C. The lower limit of a PEB time period is preferably 5 sec, more preferably 10 sec, and still more preferably 30 sec. The upper limit of the PEB time period is preferably 600 sec, more preferably 300 sec, and still more preferably 100 sec.

### Developing Step

In this step, the resist film exposed is developed. Accordingly, formation of a predetermined resist pattern is enabled. The developing is typically followed by washing with a rinse agent such as water or an alcohol, and then drying. The development procedure in the developing step may be carried out by either development with an alkali, or development with an organic solvent.

In the case of the development with an alkali, the developer solution for use in the development is exemplified by: alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (hereinafter, may be also referred to as “TMAH”), pyrrole, piperidine, choline, 1,8-diazabicyclo-[5.4.0]-7-undecene, and 1,5-diazabicyclo-[4.3.0]-5-nonene; and the like. Of these, an aqueous TMAH solution is preferred, and a 2.38% by mass aqueous TMAH solution is more preferred.

In the case of the development with an organic solvent, the developer solution is exemplified by: an organic solvent such as a hydrocarbon solvent, an ether solvent, an ester solvent, a ketone solvent, and an alcohol solvent; a solution containing the organic solvent; and the like. An exemplary organic solvent includes the solvents exemplified as the organic solvent (D) in the radiation-sensitive composition of the one embodiment of the present disclosure, and the like.

Examples of the development procedure include: a dipping procedure in which the substrate is immersed for a given time period in the developer solution charged in a container; a puddle procedure in which the developer solution is placed to form a dome-shaped bead by way of the surface tension on the surface of the substrate for a given time period to conduct a development; a spraying procedure in which the developer solution is sprayed onto the surface of the substrate; a dynamic dispensing procedure in which the developer solution is continuously discharged onto the substrate, which is rotated at a constant speed, while scanning with a developer solution-discharge nozzle at a constant speed; and the like.

Examples of the resist pattern formed by the method of forming a resist pattern include a line-and-space pattern, a contact hole pattern, and the like.

### Polymer

The polymer of still another embodiment of the present disclosure includes: a side chain having an acid-labile group; and a side chain having two or more iodo groups and one or more radiation-sensitive onium cation structure(s). The side chain having an acid-labile group is preferably included in a first structural unit which includes a partial structure obtained by substituting a hydrogen atom of a carboxy group or a hydrogen atom of a phenolic hydroxyl group with the acid-labile group. The above-described side chain having two or more iodo groups and one or more radiation-sensitive onium cation structure(s) is preferably included in a second structural unit (wherein, those corresponding to the first structural unit are excluded which includes two or more iodo groups and one or more radiation-sensitive onium cation structure(s). The constitution of the polymer is similar to that of the polymer (A) contained in the radiation-sensitive composition described above, and thus the description is incorporated here by reference.

### EXAMPLES

Hereinafter, the present invention is explained in detail by way of Examples, but the present invention is not in any way limited to these Examples. Measuring methods for various types of physical property values are shown below.

### Weight Average Molecular Weight (Mw), Number Average Molecular Weight (Mn) and Polydispersity Index (Mw/Mn)

Measurements of the Mw and the Mn of the polymer were carried out in accordance with the conditions described in the aforementioned paragraph “Method for Measuring Mw and Mn”. The polydispersity index (Mw/Mn) of the polymer was calculated from the measurement results of the Mw and the Mn.

### Synthesis of Polymer (A)

Polymers (A-1) to (A-40) and (CA-1) to (CA-3) were synthesized as the polymer (A) by a well-known method. For the synthesis of the polymer (A), compounds represented by the following formulae (M-1) to (M-16), and monomers (pm-101) to (pm-110), (pm-201) to (pm-217), (pm-301), and (pm-401) shown in Table 1 were used. In the following Synthesis Examples, unless otherwise specified particularly, the term “parts by mass” means a value, provided that the total mass of the monomer used was 100 parts by mass, and the term “mol %” means a value, provided that the total number of moles of the monomer used was 100 mol %. It is to be noted that as for pm-213, two M^{t+}s are both the cation represented by ca-1, in the compound represented by the formula (II-2-13). As for pm-216, two M^{t+}s are both the cation represented by ca-1, in the compound represented by the formula (II-2-16). Monomers other than pm-213 and pm-216 are monomers consisting of 1 mol of the cation and 1 mol of the anion.

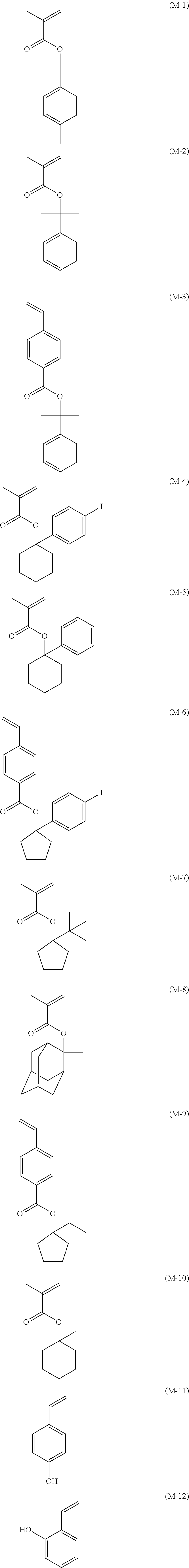

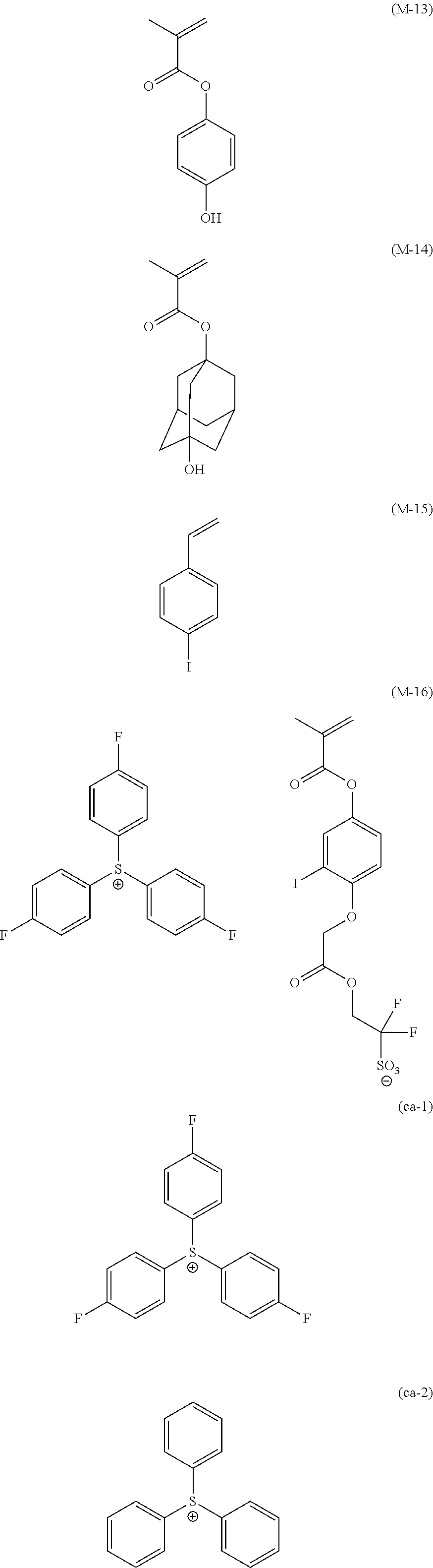

TABLE 1

| Monomer that gives | | |
|---|---|---|
| structural unit (II) | Anion | Cation |
| pm-101 | II-1-1 | ca-1 |
| pm-102 | II-1-2 | ca-1 |
| pm-103 | II-1-3 | ca-1 |
| pm-104 | II-1-4 | ca-1 |
| pm-105 | II-1-5 | ca-1 |
| pm-106 | II-1-6 | ca-1 |
| pm-107 | II-1-7 | ca-1 |
| pm-108 | II-1-8 | ca-1 |
| pm-109 | II-1-9 | ca-1 |
| pm-110 | II-1-10 | ca-1 |
| pm-201 | II-2-1 | ca-1 |
| pm-202 | II-2-2 | ca-1 |
| pm-203 | II-2-3 | ca-1 |
| pm-204 | II-2-4 | ca-1 |
| pm-205 | II-2-5 | ca-1 |
| pm-206 | II-2-6 | ca-1 |
| pm-207 | II-2-7 | ca-1 |
| pm-208 | II-2-8 | ca-1 |
| pm-209 | II-2-9 | ca-1 |
| pm-210 | II-2-10 | ca-1 |
| pm-211 | II-2-11 | ca-1 |
| pm-212 | II-2-12 | ca-1 |
| pm-213 | II-2-13 (1 mol) | ca-1 (2 mol) |
| pm-214 | II-2-14 | ca-1 |
| pm-215 | II-2-15 | ca-1 |
| pm-216 | II-2-16 (1 mol) | ca-1 (2 mol) |
| pm-217 | II-2-17 | ca-1 |
| pm-301 | II-1-1 | ca-2 |
| pm-401 | II-2-1 | ca-2 |

The type and the proportion, and the Mw and the Mw/Mn of the monomer that gives each structural unit of the polymer (A) obtained in Synthesis Examples 1 to 43 are shown in Table 2 below. It is to be noted that in Table 2 below, “-” denotes that a corresponding monomer was not used.

TABLE 2

| | | | | | | | | Monomer that | | | |
|---|---|---|---|---|---|---|---|---|---|---|---|
| | | | | | | | | gives structural | | | |
| | | Monomer that | | Monomer that | | Monomer that | | unit other than | | | |
| | | gives structural | | gives structural | | gives structural | | structural units | | | |
| | | unit (I) | | unit (II) | | unit (III) | | (I) to (III) | | | |
| | | | usage | | usage | | usage | | usage | | |
| | (A) | | amount | | amount | | amount | | amount | | Mw/ |
| | Polymer | type | (mol %) | type | (mol %) | type | (mol %) | type | (mol %) | Mw | Mn |
| Synthesis | A-1 | M-1/M-4/M-7 | 20/20/10 | pm-101 | 10 | M-11/M-12 | 10/30 | — | — | 7,400 | 1.7 |
| Example 1 | | | | | | | | | | | |
| Synthesis | A-2 | M-1/M-4/M-7 | 20/20/10 | pm-102 | 10 | M-11/M-12 | 10/30 | — | — | 7,400 | 1.7 |
| Example 2 | | | | | | | | | | | |
| Synthesis | A-3 | M-1/M-4/M-7 | 20/20/10 | pm-103 | 10 | M-11/M-12 | 10/30 | — | — | 7,300 | 1.7 |
| Example 3 | | | | | | | | | | | |
| Synthesis | A-4 | M-1/M-4/M-7 | 20/20/10 | pm-104 | 10 | M-11/M-12 | 10/30 | — | — | 8,000 | 1.7 |
| Example 4 | | | | | | | | | | | |
| Synthesis | A-5 | M-1/M-4/M-7 | 20/20/10 | pm-105 | 10 | M-11/M-12 | 10/30 | — | — | 7,800 | 1.7 |
| Example 5 | | | | | | | | | | | |
| Synthesis | A-6 | M-1/M-4/M-7 | 20/20/10 | pm-106 | 10 | M-11/M-12 | 10/30 | — | — | 7,100 | 1.8 |
| Example 6 | | | | | | | | | | | |
| Synthesis | A-7 | M-1/M-4/M-7 | 20/20/10 | pm-107 | 10 | M-11/M-12 | 10/30 | — | — | 7,400 | 1.7 |
| Example 7 | | | | | | | | | | | |
| Synthesis | A-8 | M-1/M-4/M-7 | 20/20/10 | pm-108 | 10 | M-11/M-12 | 10/30 | — | — | 7,200 | 1.7 |
| Example 8 | | | | | | | | | | | |
| Synthesis | A-9 | M-1/M-4/M-7 | 20/20/10 | pm-109 | 10 | M-11/M-12 | 10/30 | — | — | 7,000 | 1.7 |
| Example 9 | | | | | | | | | | | |
| Synthesis | A-10 | M-1/M-4/M-7 | 20/20/10 | pm-110 | 10 | M-11/M-12 | 10/30 | — | — | 8,300 | 1.6 |
| Example 10 | | | | | | | | | | | |
| Synthesis | A-11 | M-1/M-4/M-7 | 20/20/10 | pm-201 | 10 | M-11/M-12 | 10/30 | — | — | 7,900 | 1.7 |
| Example 11 | | | | | | | | | | | |
| Synthesis | A-12 | M-1/M-4/M-7 | 20/20/10 | pm-202 | 10 | M-11/M-12 | 10/30 | — | — | 7,800 | 1.7 |
| Example 12 | | | | | | | | | | | |
| Synthesis | A-13 | M-1/M-4/M-7 | 20/20/10 | pm-203 | 10 | M-11/M-12 | 10/30 | — | — | 8,400 | 1.7 |
| Example 13 | | | | | | | | | | | |
| Synthesis | A-14 | M-1/M-4/M-7 | 20/20/10 | pm-204 | 10 | M-11/M-12 | 10/30 | — | — | 8,500 | 1.7 |
| Example 14 | | | | | | | | | | | |
| Synthesis | A-15 | M-1/M-4/M-7 | 20/20/10 | pm-205 | 10 | M-11/M-12 | 10/30 | — | — | 8,400 | 1.7 |
| Example 15 | | | | | | | | | | | |
| Synthesis | A-16 | M-1/M-4/M-7 | 20/20/10 | pm-206 | 10 | M-11/M-12 | 10/30 | — | — | 8,200 | 1.7 |
| Example 16 | | | | | | | | | | | |
| Synthesis | A-17 | M-1/M-4/M-7 | 20/20/10 | pm-207 | 10 | M-11/M-12 | 10/30 | — | — | 8,000 | 1.7 |
| Example 17 | | | | | | | | | | | |
| Synthesis | A-18 | M-1/M-4/M-7 | 20/20/10 | pm-208 | 10 | M-11/M-12 | 10/30 | — | — | 7,500 | 1.8 |
| Example 18 | | | | | | | | | | | |
| Synthesis | A-19 | M-1/M-4/M-7 | 20/20/10 | pm-209 | 10 | M-11/M-12 | 10/30 | — | — | 7,800 | 1.7 |
| Example 19 | | | | | | | | | | | |
| Synthesis | A-20 | M-1/M-4/M-7 | 20/20/10 | pm-210 | 10 | M-11/M-12 | 10/30 | — | — | 7,900 | 1.7 |
| Example 20 | | | | | | | | | | | |
| Synthesis | A-21 | M-1/M-4/M-7 | 20/20/10 | pm-211 | 10 | M-11/M-12 | 10/30 | — | — | 7,600 | 1.7 |
| Example 21 | | | | | | | | | | | |
| Synthesis | A-22 | M-1/M-4/M-7 | 20/20/10 | pm-212 | 10 | M-11/M-12 | 10/30 | — | — | 7,400 | 1.8 |
| Example 22 | | | | | | | | | | | |
| Synthesis | A-23 | M-1/M-4/M-7 | 20/20/10 | pm-213 | 5 | M-11/M-12 | 10/30 | M-14 | 5 | 7,000 | 1.8 |
| Example 23 | | | | | | | | | | | |
| Synthesis | A-24 | M-1/M-4/M-7 | 20/20/10 | pm-214 | 10 | M-11/M-12 | 10/30 | — | — | 7,600 | 1.7 |
| Example 24 | | | | | | | | | | | |
| Synthesis | A-25 | M-1/M-4/M-7 | 20/20/10 | pm-215 | 10 | M-11/M-12 | 10/30 | — | — | 7,600 | 1.7 |
| Example 25 | | | | | | | | | | | |
| Synthesis | A-26 | M-1/M-4/M-7 | 20/20/10 | pm-216 | 5 | M-11/M-12 | 10/30 | M-14 | 5 | 7,000 | 1.7 |
| Example 26 | | | | | | | | | | | |
| Synthesis | A-27 | M-1/M-4/M-7 | 20/20/10 | pm-217 | 10 | M-11/M-12 | 10/30 | — | — | 7,200 | 1.6 |
| Example 27 | | | | | | | | | | | |
| Synthesis | A-28 | M-1/M-4/M-7 | 20/20/10 | pm-301 | 10 | M-11 | 40 | — | — | 7,600 | 1.7 |
| Example 28 | | | | | | | | | | | |
| Synthesis | A-29 | M-1/M-4/M-7 | 20/20/10 | pm-401 | 10 | M-12 | 40 | — | — | 8,100 | 1.7 |
| Example 29 | | | | | | | | | | | |
| Synthesis | A-30 | M-1/M-4/M-7 | 20/20/10 | pm-101 | 10 | M-13 | 40 | — | — | 7,800 | 1.7 |
| Example 30 | | | | | | | | | | | |
| Synthesis | A-31 | M-2/M-4/M-7 | 10/30/10 | pm-104 | 10 | M-11/M-12 | 10/30 | — | — | 7,400 | 1.7 |
| Example 31 | | | | | | | | | | | |
| Synthesis | A-32 | M-1/M-5/M-7 | 10/15/25 | pm-110 | 10 | M-11/M-12 | 10/30 | — | — | 7,500 | 1.7 |
| Example 32 | | | | | | | | | | | |
| Synthesis | A-33 | M-2/M-5/M-8 | 5/10/35 | pm-201 | 10 | M-11/M-12 | 10/30 | — | — | 7,900 | 1.7 |
| Example 33 | | | | | | | | | | | |
| Synthesis | A-34 | M-4/M-5 | 15/15 | pm-204 | 10 | M-11/M-12 | 10/30 | M-14 | 20 | 8,600 | 1.7 |
| Example 34 | | | | | | | | | | | |
| Synthesis | A-35 | M-1/M-2 | 25/15 | pm-207 | 10 | M-11/M-12 | 10/30 | M-14 | 10 | 8,200 | 1.7 |
| Example 35 | | | | | | | | | | | |
| Synthesis | A-36 | M-7/M-9 | 10/50 | pm-210 | 10 | M-11/M-12 | 10/20 | — | — | 8,000 | 1.7 |
| Example 36 | | | | | | | | | | | |
| Synthesis | A-37 | M-3 | 45 | pm-211 | 15 | M-11/M-12 | 10/30 | — | — | 7,800 | 1.6 |
| Example 37 | | | | | | | | | | | |
| Synthesis | A-38 | M-6/M-10 | 30/15 | pm-217 | 15 | M-11/M-12 | 10/30 | — | — | 7,300 | 1.7 |
| Example 38 | | | | | | | | | | | |
| Synthesis | A-39 | M-10 | 50 | pm-101 | 5 | M-11/M-12 | 10/30 | — | — | 8,100 | 1.6 |
| Example 39 | | | | | | | | | | | |
| Synthesis | A-40 | M-4/M-9 | 25/25 | pm-201 | 5 | M-11/M-13 | 5/25 | M-14/M-15 | 5/10 | 7,800 | 1.7 |
| Example 40 | | | | | | | | | | | |
| Synthesis | CA-1 | M-1/M-4/M-7 | 20/20/10 | — | — | M-11/M-12 | 10/30 | M-16 | 10 | 7,700 | 1.7 |
| Example 41 | | | | | | | | | | | |
| Synthesis | CA-2 | M-1/M-4/M-7 | 20/20/10 | — | — | M-11/M-12 | 10/25 | M-15/M-16 | 5/10 | 7,500 | 1.8 |
| Example 42 | | | | | | | | | | | |
| Synthesis | CA-3 | M-1/M-4/M-7 | 20/20/10 | — | — | M-11/M-12 | 10/25 | M-15 | 15 | 8,300 | 1.7 |
| Example 43 | | | | | | | | | | | |

### Preparation of Radiation-Sensitive Composition

The acid generating agent (B), the acid diffusion control agent (C), the organic solvent (D), and the polymer (F) used in preparation of the radiation-sensitive composition are shown below. In the following Examples and Comparative Examples, unless otherwise specified particularly, the term “parts by mass” means a value, provided that the mass of the polymer (A) used was 100 parts by mass, and the term “mol % o” means a value, provided that the number of moles of the acid generating agent (B) used was 100 mol %.

### (B) Acid Generating Agent

Compounds (hereinafter, may be also referred to as “acid generating agents (B-1) to (B-5)”) represented by the following formulae (B-1) to (B-5) were used as the acid generating agent (B).

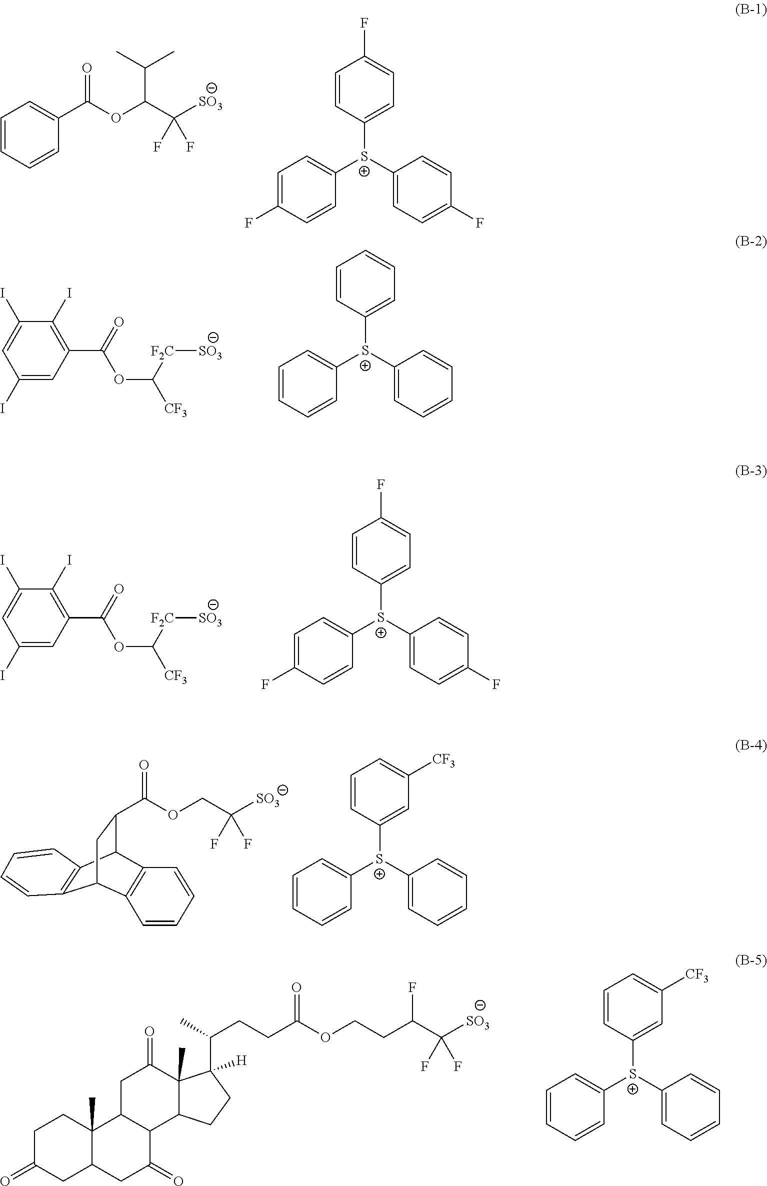

### (C) Acid Diffusion Control Agent

Compounds (hereinafter, may be also referred to as “acid diffusion control agents (C-1) to (C-4)”) represented by the following formulae (C-1) to (C-4) were used as the acid diffusion control agent (C).

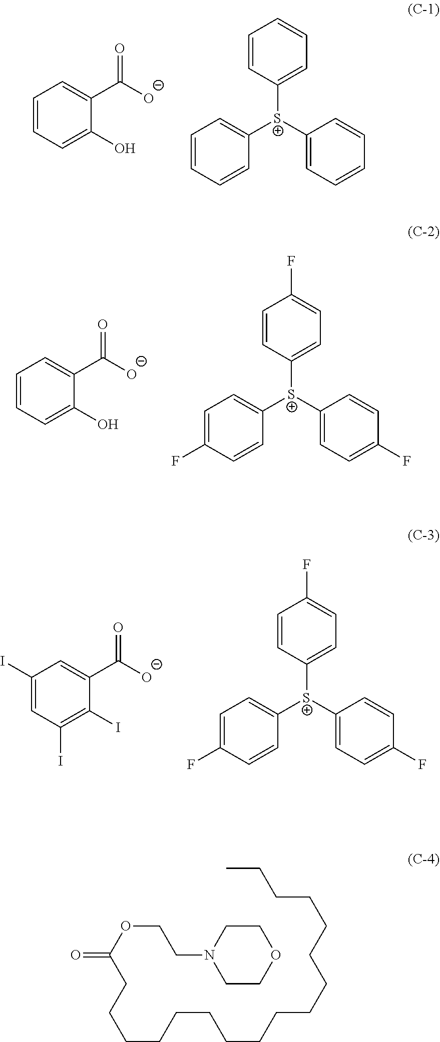

### (D) Organic Solvent

Organic solvents shown below were used as the organic solvent (D).
(D-1): propylene glycol monomethyl ether acetate
(D-2): propylene glycol monomethyl ether
(D-3): cyclohexanone
(D-4): γ-butyrolactone
(D-5): ethyl lactate
(D-6): diacetone alcohol

### (F) Polymer

A polymer represented by the following formula (F-1) was used as the polymer (F). Mw was 8,900, and Mw/Mn was 2.0.

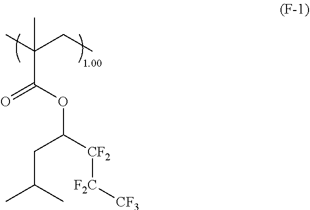

### Example 1: Preparation of Radiation-Sensitive Composition (R-1)

100 parts by mass of (A-1) as the polymer (A), 5 parts by mass of (C-1) as the acid diffusion control agent (C), and 5,000 parts by mass of (D-1) and 2,000 parts by mass of (D-2) as the organic solvent (D) were admixed. A mix liquid thus obtained was filtered through a membrane filter having a pore size of 0.20 m, whereby a radiation-sensitive composition (R-1) was prepared.

### Examples 2 to 50 and Comparative Examples 1 to 3: Preparation of Radiation-Sensitive Compositions (R-2) to (R-50) and (CR-1) to (CR-3)

Radiation-sensitive compositions (R-2) to (R-50) and (CR-1) to (CR-3) were prepared similarly to Example 1, except that each component of the following type and at the following content shown in Table 3 below was used.

### Forming of Resist Pattern

By using a spin coater (“CLEAN TRACK ACT 12,” available from Tokyo Electron Limited), each radiation-sensitive composition prepared as described above was applied on a 12-inch silicon wafer surface provided with an underlayer film (“AL412,” available from Brewer Science, Inc.) having an average thickness of 20 nm which had been formed thereon. A resist film having an average thickness of 50 nm was formed through prebaking (PB) carried out at 130° C. for 60 sec, followed by cooling at 23° C. for 30 sec. Next, this resist film was irradiated with EUV light by using an EUV scanner (“NXE3400” available from ASIL Co.: NA=0.33, quadrupole illumination conditions). After the irradiation, the resist film was subjected to post exposure baking (PEB) at 130° C. for 60 sec. Subsequently, development was performed using a 2.38% by mass aqueous TMAH solution at 23° C. for 30 sec to form a positive-tone contact hole pattern (diameter: 25 nm; 50 nm pitch).

### Evaluations

Each resist pattern formed as described above was evaluated on the sensitivity and CDU in accordance with the following methods. Line-width measurement of the resist pattern was performed using a scanning electron microscope (“CG-4100” available from Hitachi High-Tech Corporation). The results of the evaluations are shown in Table 3 below.

### Sensitivity

An exposure dose at which a contact hole pattern with a diameter of 25 nm was formed in the aforementioned resist pattern formation was defined as an optimum exposure dose, and this optimum exposure dose was adopted as Eop (mJ/cm²). A smaller sensitivity value indicates higher sensitivity, which is favorable. Using the sensitivity of Comparative Example 1 as a standard, each sensitivity is shown in Table 3 in terms of: “A” when highly sensitized by more than 6%; “B” when highly sensitized by more than 2% and 6% or less; “C” when highly sensitized by more than 0% and 2% or less; and “D” when high sensitization failed. The sensitivity of Comparative Example 1 is shown as “-”.

### CDU

The resist pattern was observed from above using the scanning electron microscope, and diameters on the contact hole pattern were measured at 800 sites in total at arbitrary locations to determine a 3 Sigma value from distribution of the measurement values and defined as “CDU” (unit: nm). A smaller CDU value indicates less variance of the hole diameters in greater ranges, which is favorable. Using the CDU of Comparative Example 1 as a standard, each CDU is shown in Table 3 in terms of: “A” when improved by more than 6%; “B” when improved by more than 2% and 6% or less; “C” when improved by more than 0% and 2% or less; and “D” when improving failed. The CDU of Comparative Example 1 is shown as “-”.

TABLE 3

| | | | | (C) Acid | | | | |
|---|---|---|---|---|---|---|---|---|
| | | | (B) Acid | diffusion | | | | |
| | Radiation- | | generating | control | (D) Organic | | | |
| | sensitive | (A) Polymer | agent | agent | solvent | (F) Polymer | | |
| | resin | (100 parts | (parts | (parts | (parts | (parts | | |
| | composition | by mass) | by mass) | by mass) | by mass) | by mass) | Sensitivity | CDU |
| Example 1 | R-1 | A-1 | — | C-1 (5) | D-1/D-2 = | — | B | B |
| | | | | | 5,000/2,000 | | | |
| Example 2 | R-2 | A-2 | — | C-1 (5) | D-1/D-2 = | — | B | B |
| | | | | | 5,000/2,000 | | | |
| Example 3 | R-3 | A-3 | — | C-1 (5) | D-1/D-2 = | — | B | B |
| | | | | | 5,000/2,000 | | | |
| Example 4 | R-4 | A-4 | — | C-1 (5) | D-1/D-2 = | — | B | B |
| | | | | | 5,000/2,000 | | | |
| Example 5 | R-5 | A-5 | — | C-1 (5) | D-1/D-2 = | — | B | B |
| | | | | | 5,000/2,000 | | | |
| Example 6 | R-6 | A-6 | — | C-1 (5) | D-1/D-2 = | — | B | B |
| | | | | | 5,000/2,000 | | | |
| Example 7 | R-7 | A-7 | — | C-1 (5) | D-1/D-2 = | — | A | B |
| | | | | | 5,000/2,000 | | | |
| Example 8 | R-8 | A-8 | — | C-1 (5) | D-1/D-2 = | — | B | B |
| | | | | | 5,000/2,000 | | | |
| Example 9 | R-9 | A-9 | — | C-1 (5) | D-1/D-2 = | — | A | B |
| | | | | | 5,000/2,000 | | | |
| Example 10 | R-10 | A-10 | — | C-1 (5) | D-1/D-2 = | — | B | B |
| | | | | | 5,000/2,000 | | | |
| Example 11 | R-11 | A-11 | — | C-1 (5) | D-1/D-2 = | — | B | A |
| | | | | | 5,000/2,000 | | | |
| Example 12 | R-12 | A-12 | — | C-1 (5) | D-1/D-2 = | — | A | A |
| | | | | | 5,000/2,000 | | | |
| Example 13 | R-13 | A-13 | — | C-1 (5) | D-1/D-2 = | — | B | A |
| | | | | | 5,000/2,000 | | | |
| Example 14 | R-14 | A-14 | — | C-1 (5) | D-1/D-2 = | — | B | A |
| | | | | | 5,000/2,000 | | | |
| Example 15 | R-15 | A-15 | — | C-1 (5) | D-1/D-2 = | — | A | A |
| | | | | | 5,000/2,000 | | | |
| Example 16 | R-16 | A-16 | — | C-1 (5) | D-1/D-2 = | — | B | A |
| | | | | | 5,000/2,000 | | | |
| Example 17 | R-17 | A-17 | — | C-1 (5) | D-1/D-2 = | — | A | A |
| | | | | | 5,000/2,000 | | | |
| Example 18 | R-18 | A-18 | — | C-1 (5) | D-1/D-2 = | — | A | A |
| | | | | | 5,000/2,000 | | | |
| Example 19 | R-19 | A-19 | — | C-1 (5) | D-1/D-2 = | — | A | A |
| | | | | | 5,000/2,000 | | | |
| Example 20 | R-20 | A-20 | — | C-1 (5) | D-1/D-2 = | — | B | A |
| | | | | | 5,000/2,000 | | | |
| Example 21 | R-21 | A-21 | — | C-1 (5) | D-1/D-2 = | — | B | A |
| | | | | | 5,000/2,000 | | | |
| Example 22 | R-22 | A-22 | — | C-1 (5) | D-1/D-2 = | — | A | A |
| | | | | | 5,000/2,000 | | | |
| Example 23 | R-23 | A-23 | — | C-1 (5) | D-1/D-2 = | — | A | A |
| | | | | | 5,000/2,000 | | | |
| Example 24 | R-24 | A-24 | — | C-1 (5) | D-1/D-2 = | — | B | A |
| | | | | | 5,000/2,000 | | | |
| Example 25 | R-25 | A-25 | — | C-1 (5) | D-1/D-2 = | — | B | A |
| | | | | | 5,000/2,000 | | | |
| Example 26 | R-26 | A-26 | — | C-1 (5) | D-1/D-2 = | — | B | A |
| | | | | | 5,000/2,000 | | | |
| Example 27 | R-27 | A-27 | — | C-1 (5) | D-1/D-2 = | — | A | A |
| | | | | | 5,000/2,000 | | | |
| Example 28 | R-28 | A-28 | — | C-1 (5) | D-1/D-2 = | — | B | B |
| | | | | | 5,000/2,000 | | | |
| Example 29 | R-29 | A-29 | — | C-1 (5) | D-1/D-2 = | — | C | A |
| | | | | | 5,000/2,000 | | | |
| Example 30 | R-30 | A-30 | — | C-1 (5) | D-1/D-2 = | — | B | B |
| | | | | | 5,000/2,000 | | | |
| Example 31 | R-31 | A-31 | — | C-1 (5) | D-1/D-2 = | — | B | B |
| | | | | | 5,000/2,000 | | | |
| Example 32 | R-32 | A-32 | — | C-1 (5) | D-1/D-2 = | — | B | B |
| | | | | | 5,000/2,000 | | | |
| Example 33 | R-33 | A-33 | — | C-1 (5) | D-1/D-2 = | — | B | A |
| | | | | | 5,000/2,000 | | | |
| Example 34 | R-34 | A-34 | — | C-1 (5) | D-1/D-2 = | — | B | A |
| | | | | | 5,000/2,000 | | | |
| Example 35 | R-35 | A-35 | — | C-1 (5) | D-1/D-2 = | — | A | A |
| | | | | | 5,000/2,000 | | | |
| Example 36 | R-36 | A-36 | — | C-1 (5) | D-1/D-2 = | — | B | A |
| | | | | | 5,000/2,000 | | | |
| Example 37 | R-37 | A-37 | — | C-1 (5) | D-1/D-2 = | — | B | A |
| | | | | | 5,000/2,000 | | | |
| Example 38 | R-38 | A-38 | — | C-1 (5) | D-1/D-2 = | — | A | A |
| | | | | | 5,000/2,000 | | | |
| Example 39 | R-39 | A-39 | — | C-1 (5) | D-1/D-2 = | — | B | B |
| | | | | | 5,000/2,000 | | | |
| Example 40 | R-40 | A-40 | — | C-1 (5) | D-1/D-2 = | — | B | A |
| | | | | | 5,000/2,000 | | | |
| Example 41 | R-41 | A-28 | — | C-1 (5) | D-1/D-2 = | F-1 (5) | B | B |
| | | | | | 5,000/2,000 | | | |
| Example 42 | R-42 | A-28 | B-1 (5) | C-1 (5) | D-1/D-2 = | — | A | B |
| | | | | | 5,000/2,000 | | | |
| Example 43 | R-43 | A-28 | B-2 (5) | C-1 (5) | D-1/D-2 = | — | B | A |
| | | | | | 5,000/2,000 | | | |
| Example 44 | R-44 | A-28 | B-3 (5) | C-1 (5) | D-1/D-2 = | — | A | A |
| | | | | | 5,000/2,000 | | | |
| Example 45 | R-45 | A-28 | — | C-2 (5) | D-1/D-2 = | — | A | B |
| | | | | | 5,000/2,000 | | | |
| Example 46 | R-46 | A-28 | — | C-3 (5) | D-1/D-2 = | — | B | A |
| | | | | | 5,000/2,000 | | | |
| Example 47 | R-47 | A-29 | B-4 (5) | C-1 (5) | D-1/D-3 = | — | C | A |
| | | | | | 5,000/2,000 | | | |
| Example 48 | R-48 | A-29 | B-5 (5) | C-1 (5) | D-1/D-4 = | — | C | A |
| | | | | | 6,000/1,000 | | | |
| Example 49 | R-49 | A-29 | — | C-1/C-4 = | D-1/D-5 = | — | C | A |
| | | | | 4/1 (5) | 6,000/1,000 | | | |
| Example 50 | R-50 | A-29 | B-3 (5) | C-3 (5) | D-1/D-6 = | — | B | A |
| | | | | | 6,000/1,000 | | | |
| Comparative | CR-1 | CA-1 | — | C-1 (5) | D-1/D-2 = | — | — | — |
| Example 1 | | | | | 5,000/2,000 | | | |
| Comparative | CR-2 | CA-2 | — | C-1 (5) | D-1/D-2 = | — | D | D |
| Example 2 | | | | | 5,000/2,000 | | | |
| Comparative | CR-3 | CA-3 | B-1 (5) | C-1 (5) | D-1/D-2 = | — | D | D |
| Example 3 | | | | | 5,000/2,000 | | | |

All the radiation-sensitive compositions of Examples were superior in the sensitivity and the CDU to the radiation-sensitive compositions of Comparative Examples. The cases of Example 42 and Example 44 in which the acid generating agents (B-1) and (B-3) each containing the onium salt compound consisting of the radiation-sensitive onium cation and the organic acid anion, wherein the radiation-sensitive onium cation includes an aromatic ring substituted with a fluorine atom were used, respectively, as the acid generating agent (B) exhibited further superior sensitivity to that of the case of Example 28 in which the acid generating agent (B-1) and/or the acid generating agent (B-3) were/was not used. The case of Example 43 in which the acid generating agent (B-2) containing the onium salt compound consisting of the radiation-sensitive onium cation and the organic acid anion, wherein the organic acid anion includes an aromatic hydrocarbon ring structure having six or more ring atoms and having one to four iodine atom(s) as substituent(s), was used as the acid generating agent (B) exhibited was further superior CDU to that of the case of Example 28 in which the acid generating agent (B-2) was not used.

In addition, the case of Example 46 in which the acid diffusion control agent (C-3) containing a compound having the monovalent radiation-sensitive onium cation and the monovalent organic acid anion, wherein a carboxylate anion which includes an aromatic ring structure having one to three iodo group(s) introduced by substitution of one to three hydrogen atom(s) is included as the organic acid anion, was used as the acid diffusion control agent (C) exhibited further superior CDU to that of the case of Example 28 in which the acid diffusion control agent (C-3) was not used.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising a polymer comprising:
an acid-labile side chain comprising an acid-labile group; and
an iodo group-containing side chain comprising two or more iodo groups and one or more radiation-sensitive onium cation structure(s).

2. The radiation-sensitive composition according to claim 1, wherein
the acid-labile side chain is included in a first structural unit comprising a partial structure obtained by substituting a hydrogen atom of a carboxy group or a hydrogen atom of a phenolic hydroxyl group with the acid-labile group, and
the iodo group-containing side chain is included in a second structural unit, which is different from the first structural unit, comprising two or more iodo groups and one or more radiation-sensitive onium cation structure(s).

3. The radiation-sensitive composition according to claim 2, wherein the second structural unit is derived from: a (meth)acrylic acid ester compound comprising two or more iodo groups and one or more radiation-sensitive onium cation structure(s); or a vinyl compound comprising two or more iodo groups and one or more radiation-sensitive onium cation structure(s).

4. The radiation-sensitive composition according to claim 3, wherein
the (meth)acrylic acid ester compound is a monomer that is a salt comprising: a sulfonate anion having a (meth)acryloyloxy group and two or more iodo groups; and a radiation-sensitive onium cation, and
the vinyl compound is a monomer that is a salt comprising: a sulfonate anion having a vinyl group and two or more iodo groups; and a radiation-sensitive onium cation.

5. The radiation-sensitive composition according to claim 1, wherein the radiation-sensitive onium cation structure comprises a monovalent radiation-sensitive onium cation comprising an aromatic ring structure which comprises an aromatic ring and comprises at least one fluorine atom bonded to the aromatic ring or at least one fluorine atom-containing group bonded to the aromatic ring.

6. The radiation-sensitive composition according to claim 1, wherein the acid-labile group is represented by formula (1-1) or (1-2): 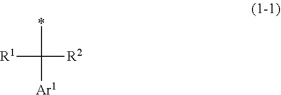
wherein
in the formula (1-1), Ar¹ represents a group obtained by removing one hydrogen atom from a substituted or unsubstituted aromatic ring structure having 5 to 30 ring atoms; R¹ and R² each independently represent a substituted or unsubstituted monovalent aliphatic hydrocarbon group having 1 to 10 carbon atoms, or R¹ and R² taken together represent a saturated alicyclic hydrocarbon ring having 3 to 8 carbon atoms together with the carbon atom to which Ar¹ bonds; and * denotes a binding site to an etheric oxygen atom of a carboxy group or an oxygen atom of a phenolic hydroxyl group 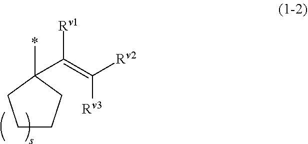
in the formula (1-2), R^{v1} to R^{v3} each independently represent a hydrogen atom or a substituted or unsubstituted monovalent chain hydrocarbon group having 1 to 10 carbon atoms; s is 1 or 2; and * denotes a binding site to an etheric oxygen atom of a carboxy group or an oxygen atom of a phenolic hydroxyl group.

7. The radiation-sensitive composition according to claim 6, wherein the substituted or unsubstituted aromatic ring structure having 5 to 30 ring atoms that gives Ar¹ in the formula (1-1) is a substituted or unsubstituted aromatic hydrocarbon ring structure having 6 to 30 ring atoms.

8. The radiation-sensitive composition according to claim 2, wherein the first structural unit is represented by following formula (3-1) or (3-2): 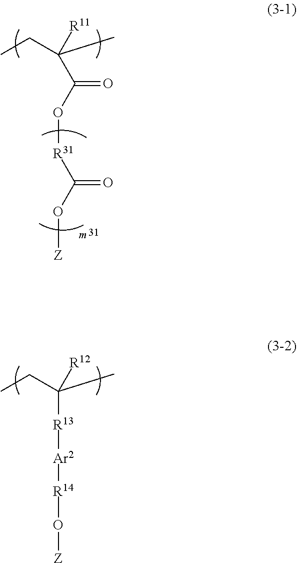
wherein
in the formulae (3-1) and (3-2), Z represents the acid-labile group;
in the formula (3-1), R¹¹ represents a hydrogen atom, a fluorine atom, a methyl group, or a trifluoromethyl group; R³¹ represents a divalent linking group; and m³¹ is 0 or 1; and
in the formula (3-2), R¹² represents a hydrogen atom or a methyl group; R¹³ represents a single bond, an oxygen atom, —COO—, or —CONH—; Ar² represents a group obtained by removing two hydrogen atoms from a substituted or unsubstituted aromatic hydrocarbon ring structure having 6 to 30 ring atoms; and R¹⁴ represents a single bond or —CO—.

9. The radiation-sensitive composition according to claim 1, wherein the polymer further comprises a side chain having a phenolic hydroxyl group.

10. The radiation-sensitive composition according to claim 1, further comprising at least one selected from the group consisting of a radiation-sensitive acid generating agent and an acid diffusion control agent.

11. A method of forming a resist pattern, the method comprising:
applying the radiation-sensitive composition according to claim 1 directly or indirectly on a substrate to form a resist film;
exposing the resist film; and
developing the resist film exposed.

12. A polymer comprising:
an acid-labile side chain comprising an acid-labile group; and
an iodo group-containing side chain comprising two or more iodo groups and one or more radiation-sensitive onium cation structure(s).

13. The polymer according to claim 12, wherein
the acid-labile side chain is included in a first structural unit comprising a partial structure obtained by substituting a hydrogen atom of a carboxy group or a hydrogen atom of a phenolic hydroxyl group with the acid-labile group, and
the iodo group-containing side chain is included in a second structural unit, which is different from the first structural unit, comprising two or more iodo groups and one or more radiation-sensitive onium cation structure(s).

14. The polymer according to claim 13, wherein the second structural unit is derived from: a (meth)acrylic acid ester compound comprising two or more iodo groups and one or more radiation-sensitive onium cation structure(s); or a vinyl compound comprising two or more iodo groups and one or more radiation-sensitive onium cation structure(s).

15. The polymer according to claim 14, wherein
the (meth)acrylic acid ester compound is a monomer that is a salt comprising: a sulfonate anion having a (meth)acryloyloxy group and two or more iodo groups; and a radiation-sensitive onium cation, and
the vinyl compound is a monomer that is a salt comprising: a sulfonate anion having a vinyl group and two or more iodo groups; and a radiation-sensitive onium cation.

16. The polymer according to claim 12, wherein the radiation-sensitive onium cation structure comprises a monovalent radiation-sensitive onium cation comprising an aromatic ring structure which comprises an aromatic ring and comprises at least one fluorine atom bonded to the aromatic ring or at least one fluorine atom-containing group bonded to the aromatic ring.

17. A monomer which is a vinyl compound comprising two or more iodo groups and one or more radiation-sensitive onium cation structure(s).

18. The monomer according to claim 17, wherein
the monomer is a salt comprising: a sulfonate anion having a vinyl group and two or more iodo groups; and a radiation-sensitive onium cation,
the monomer is a salt comprising: a sulfonate anion having a vinyl group and one iodo group; and a radiation-sensitive onium cation having one iodo group, or
the monomer is a salt comprising: a sulfonate anion having a vinyl group; and a radiation-sensitive onium cation having two or more iodo groups.

19. The monomer according to claim 17, wherein the monomer is represented by formula (II-2t) or formula (II-2u): 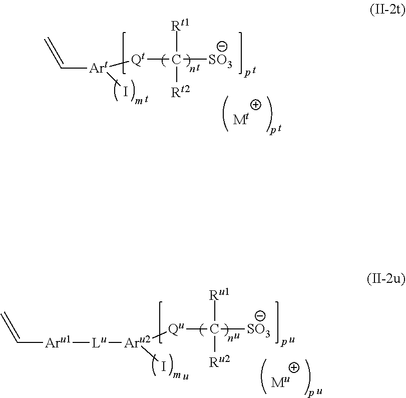
wherein
in the formula (II-2t), Q^{t} represents a single bond or a divalent linking group; Ar^{t} represents an aromatic hydrocarbon group having 6 to 20 carbon atoms and having a valency of (m^{t}+p^{t}+1); R^{t1} and R^{t2} each independently represent a fluorine atom or a monovalent fluorinated hydrocarbon group having 1 to 20 carbon atoms; m^{t} is an integer of 2 to 4; n^{t} is an integer of 1 to 10; p^{t} is 1 or 2; and M^{t+} represents a monovalent radiation-sensitive onium cation, wherein in a case in which p^{t} is 2, two Q^{t}s are identical or different and two M^{t+}s are identical or different, and in a case in which n^{t} is no less than 2 or p^{t} is 2, a plurality of included R^{t1}s and R^{t2}s are each independently identical or different.
in the formula (II-2u), L^{u} represents a divalent linking group; Q^{u} represents a single bond or a divalent linking group; Ar^{u1} represents a divalent aromatic hydrocarbon group having 6 to 20 carbon atoms; Ar^{u2} represents an aromatic hydrocarbon group having 6 to 20 carbon atoms and having a valency of (m^{u}+p^{u}+1); R^{u1} and R^{u2} each independently represent a fluorine atom or a monovalent fluorinated hydrocarbon group having 1 to 20 carbon atoms; m^{u} is an integer of 0 to 4; n^{u} is an integer of 1 to 10; p^{u} is 1 or 2; and M^{u+} represents a monovalent radiation-sensitive onium cation, wherein in a case in which p^{u} is 2, two Q^{u}s are identical or different and two M^{u+}s are identical or different, and in a case in which n^{u} is no less than 2 or p^{u} is 2, a plurality of included R^{u1}s and R^{u2}s are each independently identical or different, and wherein in a case in which m^{u} is 0, Ar^{u1} comprises an aromatic ring having two or more iodo groups, or comprises two or more aromatic rings having one iodo group, and in a case in which m^{u} is 1, Ar^{u1} comprises an aromatic ring having one or more iodo group(s).
